(19) United States
(12) Patent Application Publication
(10) Pub. No.: US 2024/0298919 A1
(43) Pub. Date: Sep. 12, 2024

(54) OXYGEN SATURATION MEASUREMENT AND REPORTING

(71) Applicant: Oura Health Oy, Oulu (FI)

(72) Inventors: Raphael Vallat, Lyon (FR); Tuomas Viljami Kenttä, Liminka (FI); Gerald Pho, Sommerville, MA (US); Xi Zhang, Daly City, CA (US); Olli-Pekka Puolitaival, Oulu (FI); Roosa Annikki Wederhorn, Espoo (FI); Ronny Li, San Francisco, CA (US); Olli Petteri Heikkinen, Oulu (FI); Tom Goff, Mountain View, CA (US); Shyamal Patel, San Francisco, CA (US)

(21) Appl. No.: 18/662,000

(22) Filed: May 13, 2024

Related U.S. Application Data
(63) Continuation-in-part of application No. 18/332,364, filed on Jun. 9, 2023.
(60) Provisional application No. 63/351,221, filed on Jun. 10, 2022.

(51) Int. Cl. A61B 5/08; A61B 5/00; A61B 5/01; A61B 5/0205; A61B 5/021; A61B 5/024; A61B 5/0245; A61B 5/11; A61B 5/1455; G16H 40/63; G16H 50/30; G16H 50/70

(52) U.S. Cl. CPC A61B 5/08; A61B 5/4812; A61B 5/6802; A61B 5/6826; A61B 5/7267; A61B 5/7285; A61B 5/743; A61B 5/7435; G16H 40/63; G16H 50/30; G16H 50/70; A61B 5/01; A61B 5/02055; A61B 5/02116; A61B 5/02427; A61B 5/0245; A61B 5/11; A61B 5/14551; A61B 2560/0242; A61B 2560/045; A61B 2560/0462; A61B 2562/0204; A61B 2562/0219; A61B 2562/0271

(57) ABSTRACT

Methods, systems, and devices for wearing detection are described. A user device may determine a condition to trigger an oxygen saturation measurement, or a measure of blood oxygen saturation (SpO2), for a user of a wearable device. The condition may be based on a physical state of the wearable device, a physiological state of the user, or both. In some examples, the user device may determine the condition based on one or more relationships between sensor data from the wearable device, application data, physiological data from the wearable device, or any combination thereof. The user device may receive a measure of oxygen saturation of the user from the wearable device. The user device may cause a graphical user interface (GUI) of the user device to display an indication of the measure of the oxygen saturation for the user.

### CROSS REFERENCE

The present Application for Patent is a Continuation-In-Part of U.S. application Ser. No. 18/332,364 by Wederhorn et al., entitled “OXYGEN SATURATION MEASUREMENT AND REPORTING,” filed Jun. 9, 2023, which claims the benefit of U.S. Provisional Patent Application No. 63/351,221 by Wederhorn et al., entitled “OXYGEN SATURATION MEASUREMENT AND REPORTING,” filed Jun. 10, 2022, assigned to the assignee hereof and expressly incorporated by reference herein.

### FIELD OF TECHNOLOGY

The following relates to wearable devices and data processing, including oxygen saturation measurement and reporting.

### BACKGROUND

Some wearable devices may be configured to collect physiological data from users, including temperature data, blood oxygen level data, heart rate data, and the like. However, the wearable device may not be triggered to collect the physiological data.

### BRIEF DESCRIPTION OF THE DRAWINGS

FIG. 1 illustrates an example of a system that supports oxygen saturation measurement and reporting in accordance with aspects of the present disclosure.

FIG. 2 illustrates an example of a system that supports oxygen saturation measurement and reporting in accordance with aspects of the present disclosure.

FIG. 3 illustrates an example of a wearable device diagram that supports oxygen saturation measurement and reporting in accordance with aspects of the present disclosure.

FIG. 4 illustrates an example of a graphical user interface (GUI) that supports oxygen saturation measurement and reporting in accordance with aspects of the present disclosure.

FIG. 5 illustrates an example of a process flow that supports oxygen saturation measurement and reporting in accordance with aspects of the present disclosure.

FIG. 6 illustrates an example of a process flow that supports breathing disturbance detection and reporting in accordance with aspects of the present disclosure.

FIG. 7 shows a block diagram of an apparatus that supports oxygen saturation measurement and reporting in accordance with aspects of the present disclosure.

FIG. 8 shows a block diagram of a wearable application that supports oxygen saturation measurement and reporting in accordance with aspects of the present disclosure.

FIG. 9 shows a diagram of a system including a device that supports oxygen saturation measurement and reporting in accordance with aspects of the present disclosure.

FIGS. 10 through 12 show flow charts illustrating methods that support oxygen saturation measurement and reporting in accordance with aspects of the present disclosure.

### DETAILED DESCRIPTION

Some wearable devices may be configured to collect data from users associated with movement and other activities. For example, some wearable devices may be configured to continuously acquire physiological data associated with a user including temperature data, heart rate data, blood oxygen level (SpO2) data, and the like. In order to efficiently and accurately track physiological data, a wearable device may be configured to collect data continuously while the user wears the device. Aspects of the disclosure are initially described in the context of systems supporting physiological data collection from users via wearable devices. However, in some cases, there may be situations in which continuously acquiring data may incur relatively high processing power at the wearable device, such as for collecting and reporting SpO2 data. For example, an SpO2 measurement may not vary significantly over time, such that continuously taking measurements may drain the battery of the wearable device without providing meaningful data.

Accordingly, techniques described herein are directed to systems and methods for measuring and reporting SpO2, or oxygen saturation, at a wearable device based on a trigger. More specifically, techniques described herein are directed to a user device determining a condition to trigger an SpO2 measurement, such as a physical state of the wearable device, a physiological state of a user of the user device, or both. The triggering condition may be based on one or more relationships between sensor data from the wearable device, application data, physiological data from the wearable device, or any combination thereof. That is, if a position, orientation, pressure and/or orientation of the wearable device is sufficient for accurate SpO2 measurements, the user device may indicate for the wearable device to perform the SpO2 measurement. Similarly, if a heart rate of a user, an activity of the user, the location of the user, a breathing rate of the user, or any combination thereof is sufficient for an accurate SpO2 measurement, the user device may indicate for the wearable device to perform the SpO2 measurement. The wearable device may report the triggered SpO2 measurement to the user device for display on a graphical user interface (GUI).

In some examples, techniques described herein may be used to determine a breathing quality of a user (e.g., during a sleep stage of the user), such as a breathing quality related to sleep apnea. As described herein, sleep apnea may be characterized by a partial or complete closure of an airway of a user (e.g., during a sleep stage of the user). Sleep apnea (e.g., or other sleep-related breathing disturbance) may result in snoring, choking, and/or breathing pauses during sleep associated with decreased oxygen levels and/or cortical arousal. In some examples, sleep apnea or other breathing disturbances may result in daytime manifestations such as excessive sleepiness, morning headaches, mood changes, and erectile dysfunction. In some examples, individuals experiencing sleep apnea may not be diagnosed, as some primary care settings may not screen for sleep apnea.

Accordingly, as described herein, a system including a wearable ring device and a user device may determine to measure an SpO2 of the user (e.g., based on SpO2 triggering techniques described herein), and may use the SpO2 measurements and/or one or more other physiological measurements (e.g., photoplethysmogram (PPG), motion, temperature, and/or audio measurements) to determine the breathing quality of the user. The system may input one or more of the physiological measurements into a neural network, which may output a breathing quality metric such as a probability that the user experiences apnea, hypopnea, oxygen desaturation, or respiratory effort-related arousal (RERA). The system may determine an index (e.g., a breathing disturbance index (BDI), an apnea-hypopnea index (AHI), an oxygen desaturation index (ODI), a hypoxic burden (HB)) associated with the breathing quality, and may display the index via the GUI.

In some examples, wearable sleep trackers (e.g., a wearable ring device) may enable prolonged physiological data collection through continuous monitoring capabilities. The wearable sleep trackers may capture comprehensive sleep patterns and potential irregularities associated with sleep apnea, such as pauses in breathing. Wearable devices may detect changes indicative of respiratory disturbances during a sleep phase of the user via sensors configured for heart rate, oxygen saturation, movement, temperature, and/or audio. Furthermore, users may collect breathing-disturbance related information at home (e.g., as opposed to a doctor's office), which may enable more accessible sleep apnea detection. In some examples, a quality of measurements (e.g., signal strength) of PPG measurements and temperature measurements may be relatively higher for a wearable ring device than for some other wearable devices (e.g., wrist-worn wearable devices). Additionally, a wearable ring device may have a relatively more comfortable form factor than some other wearable devices (e.g., due to tightness around a finger of the user rather than a wrist), which may increase user experience.

Aspects of the disclosure are initially described in the context of systems supporting physiological data collection from users via wearable devices. Additional aspects of the disclosure are described in the context of wearable user device diagrams, an example GUI, and process flows. Aspects of the disclosure are further illustrated by and described with reference to apparatus diagrams, system diagrams, and flowcharts that relate to oxygen saturation measurement and reporting.

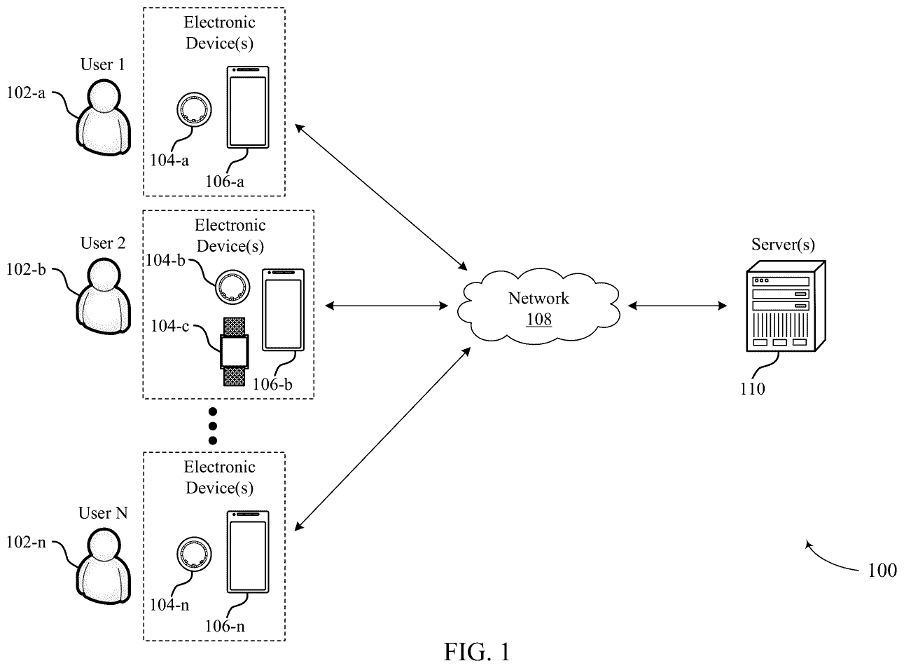

FIG. 1 illustrates an example of a system 100 that supports oxygen saturation measurement and reporting in accordance with aspects of the present disclosure. The system 100 includes a plurality of electronic devices (e.g., wearable devices 104, user devices 106) that may be worn and/or operated by one or more users 102. The system 100 further includes a network 108 and one or more servers 110.

The electronic devices may include any electronic devices known in the art, including wearable devices 104 (e.g., ring wearable devices, watch wearable devices, etc.), user devices 106 (e.g., smartphones, laptops, tablets). The electronic devices associated with the respective users 102 may include one or more of the following functionalities: 1) measuring physiological data, 2) storing the measured data, 3) processing the data, 4) providing outputs (e.g., via GUIs) to a user 102 based on the processed data, and 5) communicating data with one another and/or other computing devices. Different electronic devices may perform one or more of the functionalities.

Example wearable devices 104 may include wearable computing devices, such as a ring computing device (hereinafter “ring”) configured to be worn on a user's 102 finger, a wrist computing device (e.g., a smart watch, fitness band, or bracelet) configured to be worn on a user's 102 wrist, and/or a head mounted computing device (e.g., glasses/goggles). Wearable devices 104 may also include bands, straps (e.g., flexible or inflexible bands or straps), stick-on sensors, and the like, that may be positioned in other locations, such as bands around the head (e.g., a forehead headband), arm (e.g., a forearm band and/or bicep band), and/or leg (e.g., a thigh or calf band), behind the car, under the armpit, and the like. Wearable devices 104 may also be attached to, or included in, articles of clothing. For example, wearable devices 104 may be included in pockets and/or pouches on clothing. As another example, wearable device 104 may be clipped and/or pinned to clothing, or may otherwise be maintained within the vicinity of the user 102. Example articles of clothing may include, but are not limited to, hats, shirts, gloves, pants, socks, outerwear (e.g., jackets), and undergarments. In some implementations, wearable devices 104 may be included with other types of devices such as training/sporting devices that are used during physical activity. For example, wearable devices 104 may be attached to, or included in, a bicycle, skis, a tennis racket, a golf club, and/or training weights.

Much of the present disclosure may be described in the context of a ring wearable device 104. Accordingly, the terms “ring 104,” “wearable device 104,” and like terms, may be used interchangeably, unless noted otherwise herein. However, the use of the term “ring 104” is not to be regarded as limiting, as it is contemplated herein that aspects of the present disclosure may be performed using other wearable devices (e.g., watch wearable devices, necklace wearable device, bracelet wearable devices, earring wearable devices, anklet wearable devices, and the like).

In some aspects, user devices 106 may include handheld mobile computing devices, such as smartphones and tablet computing devices. User devices 106 may also include personal computers, such as laptop and desktop computing devices. Other example user devices 106 may include server computing devices that may communicate with other electronic devices (e.g., via the Internet). In some implementations, computing devices may include medical devices, such as external wearable computing devices (e.g., Holter monitors). Medical devices may also include implantable medical devices, such as pacemakers and cardioverter defibrillators. Other example user devices 106 may include home computing devices, such as internet of things (IoT) devices (e.g., IoT devices), smart televisions, smart speakers, smart displays (e.g., video call displays), hubs (e.g., wireless communication hubs), security systems, smart appliances (e.g., thermostats and refrigerators), and fitness equipment.

Some electronic devices (e.g., wearable devices 104, user devices 106) may measure physiological parameters of respective users 102, such as photoplethysmography waveforms, continuous skin temperature, a pulse waveform, respiration rate, heart rate, heart rate variability (HRV), actigraphy, galvanic skin response, pulse oximetry, and/or other physiological parameters. Some electronic devices that measure physiological parameters may also perform some/all of the calculations described herein. Some electronic devices may not measure physiological parameters, but may perform some/all of the calculations described herein. For example, a ring (e.g., wearable device 104), mobile device application, or a server computing device may process received physiological data that was measured by other devices.

In some implementations, a user 102 may operate, or may be associated with, multiple electronic devices, some of which may measure physiological parameters and some of which may process the measured physiological parameters. In some implementations, a user 102 may have a ring (e.g., wearable device 104) that measures physiological parameters. The user 102 may also have, or be associated with, a user device 106 (e.g., mobile device, smartphone), where the wearable device 104 and the user device 106 are communicatively coupled to one another. In some cases, the user device 106 may receive data from the wearable device 104 and perform some/all of the calculations described herein. In some implementations, the user device 106 may also measure physiological parameters described herein, such as motion/activity parameters.

For example, as illustrated in FIG. 1, a first user 102-a (User 1) may operate, or may be associated with, a wearable device 104-a (e.g., ring 104-a) and a user device 106-a that may operate as described herein. In this example, the user device 106-a associated with user 102-a may process/store physiological parameters measured by the ring 104-a. Comparatively, a second user 102-b (User 2) may be associated with a ring 104-b, a watch wearable device 104-c (e.g., watch 104-c), and a user device 106-b, where the user device 106-b associated with user 102-b may process/store physiological parameters measured by the ring 104-b and/or the watch 104-c. Moreover, an nth user 102-n (User N) may be associated with an arrangement of electronic devices described herein (e.g., ring 104-n, user device 106-n). In some aspects, wearable devices 104 (e.g., rings 104, watches 104) and other electronic devices may be communicatively coupled to the user devices 106 of the respective users 102 via Bluetooth, Wi-Fi, and other wireless protocols.

In some implementations, the rings 104 (e.g., wearable devices 104) of the system 100 may be configured to collect physiological data from the respective users 102 based on arterial blood flow within the user's finger. In particular, a ring 104 may utilize one or more LEDs (e.g., red LEDs, green LEDs) that emit light on the palm-side of a user's finger to collect physiological data based on arterial blood flow within the user's finger. In some cases, the system 100 may be configured to collect physiological data from the respective users 102 based on blood flow diffused into a microvascular bed of skin with capillaries and arterioles. For example, the system 100 may collect PPG data based on a measured amount of blood diffused into the microvascular system of capillaries and arterioles. In some implementations, the ring 104 may acquire the physiological data using a combination of both green and red LEDs. The physiological data may include any physiological data known in the art including, but not limited to, temperature data, accelerometer data (e.g., movement/motion data), heart rate data, HRV data, blood oxygen level data, or any combination thereof.

The use of both green and red LEDs may provide several advantages over other solutions, as red and green LEDs have been found to have their own distinct advantages when acquiring physiological data under different conditions (e.g., light/dark, active/inactive) and via different parts of the body, and the like. For example, green LEDs have been found to exhibit better performance during exercise. Moreover, using multiple LEDs (e.g., green and red LEDs) distributed around the ring 104 has been found to exhibit superior performance as compared to wearable devices that utilize LEDs that are positioned close to one another, such as within a watch wearable device. Furthermore, the blood vessels in the finger (e.g., arteries, capillaries) are more accessible via LEDs as compared to blood vessels in the wrist. In particular, arteries in the wrist are positioned on the bottom of the wrist (e.g., palm-side of the wrist), meaning only capillaries are accessible on the top of the wrist (e.g., back of hand side of the wrist), where wearable watch devices and similar devices are typically worn. As such, utilizing LEDs and other sensors within a ring 104 has been found to exhibit superior performance as compared to wearable devices worn on the wrist, as the ring 104 may have greater access to arteries (as compared to capillaries), thereby resulting in stronger signals and more valuable physiological data.

The electronic devices of the system 100 (e.g., user devices 106, wearable devices 104) may be communicatively coupled to one or more servers 110 via wired or wireless communication protocols. For example, as shown in FIG. 1, the electronic devices (e.g., user devices 106) may be communicatively coupled to one or more servers 110 via a network 108. The network 108 may implement transfer control protocol and internet protocol (TCP/IP), such as the Internet, or may implement other network 108 protocols. Network connections between the network 108 and the respective electronic devices may facilitate transport of data via email, web, text messages, mail, or any other appropriate form of interaction within a computer network 108. For example, in some implementations, the ring 104-a associated with the first user 102-a may be communicatively coupled to the user device 106-a, where the user device 106-a is communicatively coupled to the servers 110 via the network 108. In additional or alternative cases, wearable devices 104 (e.g., rings 104, watches 104) may be directly communicatively coupled to the network 108.

The system 100 may offer an on-demand database service between the user devices 106 and the one or more servers 110. In some cases, the servers 110 may receive data from the user devices 106 via the network 108, and may store and analyze the data. Similarly, the servers 110 may provide data to the user devices 106 via the network 108. In some cases, the servers 110 may be located at one or more data centers. The servers 110 may be used for data storage, management, and processing. In some implementations, the servers 110 may provide a web-based interface to the user device 106 via web browsers.

In some aspects, the system 100 may detect periods of time during which a user 102 is asleep, and classify periods of time during which the user 102 is asleep into one or more sleep stages (e.g., sleep stage classification). For example, as shown in FIG. 1, User 102-a may be associated with a wearable device 104-a (e.g., ring 104-a) and a user device 106-a. In this example, the ring 104-a may collect physiological data associated with the user 102-a, including temperature, heart rate, HRV, respiratory rate, and the like. In some aspects, data collected by the ring 104-a may be input to a machine learning classifier, where the machine learning classifier is configured to determine periods of time during which the user 102-a is (or was) asleep. Moreover, the machine learning classifier may be configured to classify periods of time into different sleep stages, including an awake sleep stage, a rapid eye movement (REM) sleep stage, a light sleep stage (non-REM (NREM)), and a deep sleep stage (NREM). In some aspects, the classified sleep stages may be displayed to the user 102-a via a GUI of the user device 106-a. Sleep stage classification may be used to provide feedback to a user 102-a regarding the user's sleeping patterns, such as recommended bedtimes, recommended wake-up times, and the like. Moreover, in some implementations, sleep stage classification techniques described herein may be used to calculate scores for the respective user, such as Sleep Scores, Readiness Scores, and the like.

In some aspects, the system 100 may utilize circadian rhythm-derived features to further improve physiological data collection, data processing procedures, and other techniques described herein. The term circadian rhythm may refer to a natural, internal process that regulates an individual's sleep-wake cycle, that repeats approximately every 24 hours. In this regard, techniques described herein may utilize circadian rhythm adjustment models to improve physiological data collection, analysis, and data processing. For example, a circadian rhythm adjustment model may be input into a machine learning classifier along with physiological data collected from the user 102-a via the wearable device 104-a. In this example, the circadian rhythm adjustment model may be configured to “weight,” or adjust, physiological data collected throughout a user's natural, approximately 24-hour circadian rhythm. In some implementations, the system may initially start with a “baseline” circadian rhythm adjustment model, and may modify the baseline model using physiological data collected from each user 102 to generate tailored, individualized circadian rhythm adjustment models that are specific to each respective user 102.

In some aspects, the system 100 may utilize other biological rhythms to further improve physiological data collection, analysis, and processing by phase of these other rhythms. For example, if a weekly rhythm is detected within an individual's baseline data, then the model may be configured to adjust “weights” of data by day of the week. Biological rhythms that may require adjustment to the model by this method include: 1) ultradian (faster than a day rhythms, including sleep cycles in a sleep state, and oscillations from less than an hour to several hours periodicity in the measured physiological variables during wake state; 2) circadian rhythms; 3) non-endogenous daily rhythms shown to be imposed on top of circadian rhythms, as in work schedules; 4) weekly rhythms, or other artificial time periodicities exogenously imposed (e.g., in a hypothetical culture with 12 day “weeks”, 12 day rhythms could be used); 5) multi-day ovarian rhythms in women and spermatogenesis rhythms in men; 6) lunar rhythms (relevant for individuals living with low or no artificial lights); and 7) seasonal rhythms.

The biological rhythms are not always stationary rhythms. For example, many women experience variability in ovarian cycle length across cycles, and ultradian rhythms are not expected to occur at exactly the same time or periodicity across days even within a user. As such, signal processing techniques sufficient to quantify the frequency composition while preserving temporal resolution of these rhythms in physiological data may be used to improve detection of these rhythms, to assign phase of each rhythm to each moment in time measured, and to thereby modify adjustment models and comparisons of time intervals. The biological rhythm-adjustment models and parameters can be added in linear or non-linear combinations as appropriate to more accurately capture the dynamic physiological baselines of an individual or group of individuals.

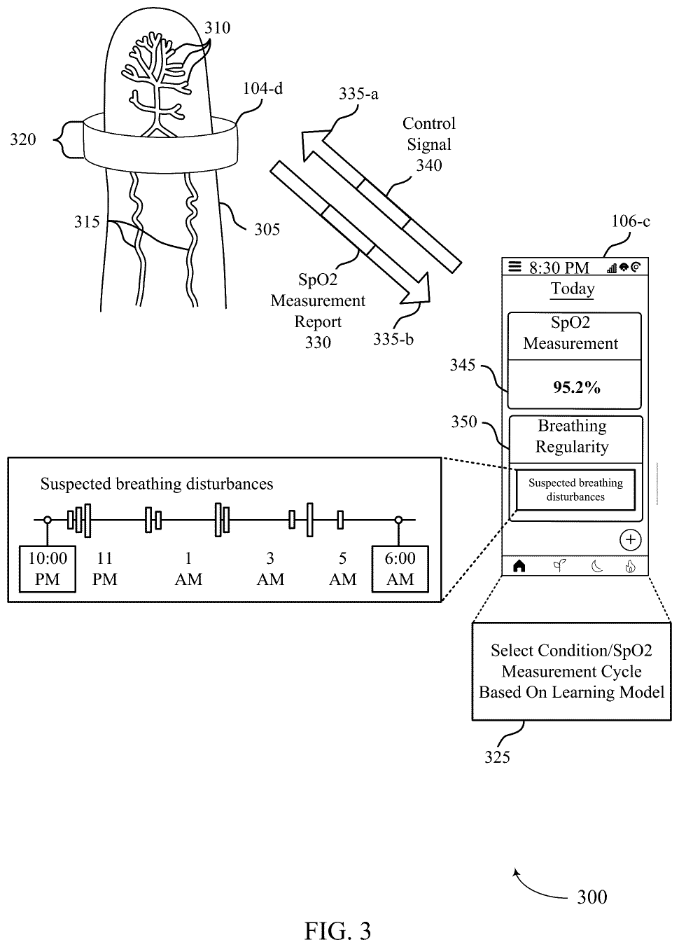

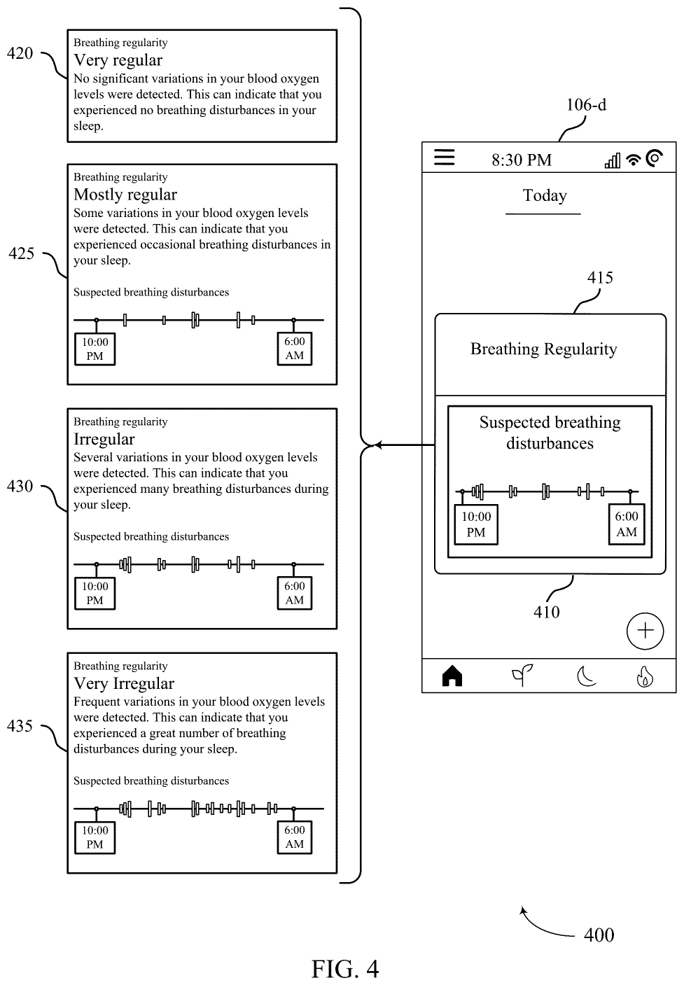

In some aspects, the respective devices of the system 100 may support techniques for triggering an SpO2, or oxygen saturation, measurement at a wearable device 104 based on a triggering condition. Specifically, techniques described herein support selecting a triggering condition at a user device 106, and indicating to the wearable device to perform the SpO2 measurement in accordance with the triggering condition. In some examples, the triggering condition may be based on accuracy of the SpO2 measurement, such as a position, orientation, pressure, heart rate data reading, respiratory rate reading, location of a user 102, activity performed by the user 102, or the like. For example, if the user 102 positions the wearable device 104 at a location or position where the SpO2 measurements may be relatively accurate, such as at a tip of a finger if the wearable device 104 is a ring, the triggering condition may be met, and the wearable device 104 may perform the SpO2 measurement. The wearable device 104 may report the SpO2 measurement to the user device 106 in signaling, which is described in further detail with respect to FIG. 3. The user device 106 may display the collected SpO2 measurement, the triggering condition, or both to the user 102 via a GUI at the user device 106, which is described in further detail with respect to FIGS. 3 and 4.

It should be appreciated by a person skilled in the art that one or more aspects of the disclosure may be implemented in a system 100 to additionally or alternatively solve other problems than those described above. Furthermore, aspects of the disclosure may provide technical improvements to “conventional” systems or processes as described herein. However, the description and appended drawings only include example technical improvements resulting from implementing aspects of the disclosure, and accordingly do not represent all of the technical improvements provided within the scope of the claims.

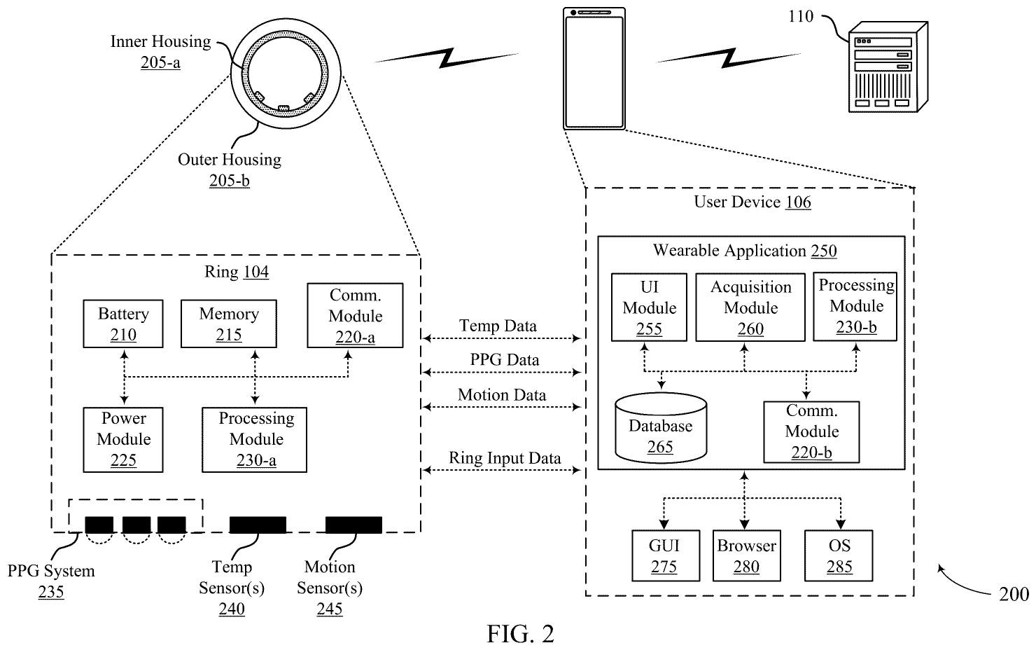

FIG. 2 illustrates an example of a system 200 that supports oxygen saturation measurement and reporting in accordance with aspects of the present disclosure. The system 200 may implement, or be implemented by, system 100. In particular, system 200 illustrates an example of a ring 104 (e.g., wearable device 104), a user device 106, and a server 110, as described with reference to FIG. 1.

In some aspects, the ring 104 may be configured to be worn around a user's finger, and may determine one or more user physiological parameters when worn around the user's finger. Example measurements and determinations may include, but are not limited to, user skin temperature, pulse waveforms, respiratory rate, heart rate, HRV, blood oxygen levels, and the like.

The system 200 further includes a user device 106 (e.g., a smartphone) in communication with the ring 104. For example, the ring 104 may be in wireless and/or wired communication with the user device 106. In some implementations, the ring 104 may send measured and processed data (e.g., temperature data, photoplethysmogram (PPG) data, motion/accelerometer data, ring input data, and the like) to the user device 106. The user device 106 may also send data to the ring 104, such as ring 104 firmware/configuration updates. The user device 106 may process data. In some implementations, the user device 106 may transmit data to the server 110 for processing and/or storage.

The ring 104 may include a housing 205 that may include an inner housing 205-a and an outer housing 205-b. In some aspects, the housing 205 of the ring 104 may store or otherwise include various components of the ring including, but not limited to, device electronics, a power source (e.g., battery 210, and/or capacitor), one or more substrates (e.g., printable circuit boards) that interconnect the device electronics and/or power source, and the like. The device electronics may include device modules (e.g., hardware/software), such as: a processing module 230-a, a memory 215, a communication module 220-a, a power module 225, and the like. The device electronics may also include one or more sensors. Example sensors may include one or more temperature sensors 240, a PPG sensor assembly (e.g., PPG system 235), and one or more motion sensors 245.

The sensors may include associated modules (not illustrated) configured to communicate with the respective components/modules of the ring 104, and generate signals associated with the respective sensors. In some aspects, each of the components/modules of the ring 104 may be communicatively coupled to one another via wired or wireless connections. Moreover, the ring 104 may include additional and/or alternative sensors or other components that are configured to collect physiological data from the user, including light sensors (e.g., LEDs), oximeters, and the like.

The ring 104 shown and described with reference to FIG. 2 is provided solely for illustrative purposes. As such, the ring 104 may include additional or alternative components as those illustrated in FIG. 2. Other rings 104 that provide functionality described herein may be fabricated. For example, rings 104 with fewer components (e.g., sensors) may be fabricated. In a specific example, a ring 104 with a single temperature sensor 240 (or other sensor), a power source, and device electronics configured to read the single temperature sensor 240 (or other sensor) may be fabricated. In another specific example, a temperature sensor 240 (or other sensor) may be attached to a user's finger (e.g., using a clamps, spring loaded clamps, etc.). In this case, the sensor may be wired to another computing device, such as a wrist worn computing device that reads the temperature sensor 240 (or other sensor). In other examples, a ring 104 that includes additional sensors and processing functionality may be fabricated.

The housing 205 may include one or more housing 205 components. The housing 205 may include an outer housing 205-b component (e.g., a shell) and an inner housing 205-a component (e.g., a molding). The housing 205 may include additional components (e.g., additional layers) not explicitly illustrated in FIG. 2. For example, in some implementations, the ring 104 may include one or more insulating layers that electrically insulate the device electronics and other conductive materials (e.g., electrical traces) from the outer housing 205-b (e.g., a metal outer housing 205-b). The housing 205 may provide structural support for the device electronics, battery 210, substrate(s), and other components. For example, the housing 205 may protect the device electronics, battery 210, and substrate(s) from mechanical forces, such as pressure and impacts. The housing 205 may also protect the device electronics, battery 210, and substrate(s) from water and/or other chemicals.

The outer housing 205-b may be fabricated from one or more materials. In some implementations, the outer housing 205-b may include a metal, such as titanium, that may provide strength and abrasion resistance at a relatively light weight. The outer housing 205-b may also be fabricated from other materials, such polymers. In some implementations, the outer housing 205-b may be protective as well as decorative.

The inner housing 205-a may be configured to interface with the user's finger. The inner housing 205-a may be formed from a polymer (e.g., a medical grade polymer) or other material. In some implementations, the inner housing 205-a may be transparent. For example, the inner housing 205-a may be transparent to light emitted by the PPG light emitting diodes (LEDs). In some implementations, the inner housing 205-a component may be molded onto the outer housing 205-a. For example, the inner housing 205-a may include a polymer that is molded (e.g., injection molded) to fit into an outer housing 205-b metallic shell.

The ring 104 may include one or more substrates (not illustrated). The device electronics and battery 210 may be included on the one or more substrates. For example, the device electronics and battery 210 may be mounted on one or more substrates. Example substrates may include one or more printed circuit boards (PCBs), such as flexible PCB (e.g., polyimide). In some implementations, the electronics/battery 210 may include surface mounted devices (e.g., surface-mount technology (SMT) devices) on a flexible PCB. In some implementations, the one or more substrates (e.g., one or more flexible PCBs) may include electrical traces that provide electrical communication between device electronics. The electrical traces may also connect the battery 210 to the device electronics.

The device electronics, battery 210, and substrates may be arranged in the ring 104 in a variety of ways. In some implementations, one substrate that includes device electronics may be mounted along the bottom of the ring 104 (e.g., the bottom half), such that the sensors (e.g., PPG system 235, temperature sensors 240, motion sensors 245, and other sensors) interface with the underside of the user's finger. In these implementations, the battery 210 may be included along the top portion of the ring 104 (e.g., on another substrate).

The various components/modules of the ring 104 represent functionality (e.g., circuits and other components) that may be included in the ring 104. Modules may include any discrete and/or integrated electronic circuit components that implement analog and/or digital circuits capable of producing the functions attributed to the modules herein. For example, the modules may include analog circuits (e.g., amplification circuits, filtering circuits, analog/digital conversion circuits, and/or other signal conditioning circuits). The modules may also include digital circuits (e.g., combinational or sequential logic circuits, memory circuits etc.).

The memory 215 (memory module) of the ring 104 may include any volatile, non-volatile, magnetic, or electrical media, such as a random access memory (RAM), read-only memory (ROM), non-volatile RAM (NVRAM), electrically-erasable programmable ROM (EEPROM), flash memory, or any other memory device. The memory 215 may store any of the data described herein. For example, the memory 215 may be configured to store data (e.g., motion data, temperature data, PPG data) collected by the respective sensors and PPG system 235. Furthermore, memory 215 may include instructions that, when executed by one or more processing circuits, cause the modules to perform various functions attributed to the modules herein. The device electronics of the ring 104 described herein are only example device electronics. As such, the types of electronic components used to implement the device electronics may vary based on design considerations.

The functions attributed to the modules of the ring 104 described herein may be embodied as one or more processors, hardware, firmware, software, or any combination thereof. Depiction of different features as modules is intended to highlight different functional aspects and does not necessarily imply that such modules must be realized by separate hardware/software components. Rather, functionality associated with one or more modules may be performed by separate hardware/software components or integrated within common hardware/software components.

The processing module 230-a of the ring 104 may include one or more processors (e.g., processing units), microcontrollers, digital signal processors, systems on a chip (SOCs), and/or other processing devices. The processing module 230-a communicates with the modules included in the ring 104. For example, the processing module 230-a may transmit/receive data to/from the modules and other components of the ring 104, such as the sensors. As described herein, the modules may be implemented by various circuit components. Accordingly, the modules may also be referred to as circuits (e.g., a communication circuit and power circuit).

The processing module 230-a may communicate with the memory 215. The memory 215 may include computer-readable instructions that, when executed by the processing module 230-a, cause the processing module 230-a to perform the various functions attributed to the processing module 230-a herein. In some implementations, the processing module 230-a (e.g., a microcontroller) may include additional features associated with other modules, such as communication functionality provided by the communication module 220-a (e.g., an integrated Bluetooth Low Energy transceiver) and/or additional onboard memory 215.

The communication module 220-a may include circuits that provide wireless and/or wired communication with the user device 106 (e.g., communication module 220-b of the user device 106). In some implementations, the communication modules 220-a, 220-b may include wireless communication circuits, such as Bluetooth circuits and/or Wi-Fi circuits. In some implementations, the communication modules 220-a, 220-b can include wired communication circuits, such as Universal Serial Bus (USB) communication circuits. Using the communication module 220-a, the ring 104 and the user device 106 may be configured to communicate with each other. The processing module 230-a of the ring may be configured to transmit/receive data to/from the user device 106 via the communication module 220-a. Example data may include, but is not limited to, motion data, temperature data, pulse waveforms, heart rate data, HRV data, PPG data, and status updates (e.g., charging status, battery charge level, and/or ring 104 configuration settings). The processing module 230-a of the ring may also be configured to receive updates (e.g., software/firmware updates) and data from the user device 106.

The ring 104 may include a battery 210 (e.g., a rechargeable battery 210). An example battery 210 may include a Lithium-Ion or Lithium-Polymer type battery 210, although a variety of battery 210 options are possible. The battery 210 may be wirelessly charged. In some implementations, the ring 104 may include a power source other than the battery 210, such as a capacitor. The power source (e.g., battery 210 or capacitor) may have a curved geometry that matches the curve of the ring 104. In some aspects, a charger or other power source may include additional sensors that may be used to collect data in addition to, or which supplements, data collected by the ring 104 itself. Moreover, a charger or other power source for the ring 104 may function as a user device 106, in which case the charger or other power source for the ring 104 may be configured to receive data from the ring 104, store and/or process data received from the ring 104, and communicate data between the ring 104 and the servers 110.

In some aspects, the ring 104 includes a power module 225 that may control charging of the battery 210. For example, the power module 225 may interface with an external wireless charger that charges the battery 210 when interfaced with the ring 104. The charger may include a datum structure that mates with a ring 104 datum structure to create a specified orientation with the ring 104 during charging. The power module 225 may also regulate voltage(s) of the device electronics, regulate power output to the device electronics, and monitor the state of charge of the battery 210. In some implementations, the battery 210 may include a protection circuit module (PCM) that protects the battery 210 from high current discharge, over voltage during charging, and under voltage during discharge. The power module 225 may also include electro-static discharge (ESD) protection.

The one or more temperature sensors 240 may be electrically coupled to the processing module 230-a. The temperature sensor 240 may be configured to generate a temperature signal (e.g., temperature data) that indicates a temperature read or sensed by the temperature sensor 240. The processing module 230-a may determine a temperature of the user in the location of the temperature sensor 240. For example, in the ring 104, temperature data generated by the temperature sensor 240 may indicate a temperature of a user at the user's finger (e.g., skin temperature). In some implementations, the temperature sensor 240 may contact the user's skin. In other implementations, a portion of the housing 205 (e.g., the inner housing 205-a) may form a barrier (e.g., a thin, thermally conductive barrier) between the temperature sensor 240 and the user's skin. In some implementations, portions of the ring 104 configured to contact the user's finger may have thermally conductive portions and thermally insulative portions. The thermally conductive portions may conduct heat from the user's finger to the temperature sensors 240. The thermally insulative portions may insulate portions of the ring 104 (e.g., the temperature sensor 240) from ambient temperature.

In some implementations, the temperature sensor 240 may generate a digital signal (e.g., temperature data) that the processing module 230-a may use to determine the temperature. As another example, in cases where the temperature sensor 240 includes a passive sensor, the processing module 230-a (or a temperature sensor 240 module) may measure a current/voltage generated by the temperature sensor 240 and determine the temperature based on the measured current/voltage. Example temperature sensors 240 may include a thermistor, such as a negative temperature coefficient (NTC) thermistor, or other types of sensors including resistors, transistors, diodes, and/or other electrical/electronic components.

The processing module 230-a may sample the user's temperature over time. For example, the processing module 230-a may sample the user's temperature according to a sampling rate. An example sampling rate may include one sample per second, although the processing module 230-a may be configured to sample the temperature signal at other sampling rates that are higher or lower than one sample per second. In some implementations, the processing module 230-a may sample the user's temperature continuously throughout the day and night. Sampling at a sufficient rate (e.g., one sample per second) throughout the day may provide sufficient temperature data for analysis described herein.

The processing module 230-a may store the sampled temperature data in memory 215. In some implementations, the processing module 230-a may process the sampled temperature data. For example, the processing module 230-a may determine average temperature values over a period of time. In one example, the processing module 230-a may determine an average temperature value each minute by summing all temperature values collected over the minute and dividing by the number of samples over the minute. In a specific example where the temperature is sampled at one sample per second, the average temperature may be a sum of all sampled temperatures for one minute divided by sixty seconds. The memory 215 may store the average temperature values over time. In some implementations, the memory 215 may store average temperatures (e.g., one per minute) instead of sampled temperatures in order to conserve memory 215.

The sampling rate, which may be stored in memory 215, may be configurable. In some implementations, the sampling rate may be the same throughout the day and night. In other implementations, the sampling rate may be changed throughout the day/night. In some implementations, the ring 104 may filter/reject temperature readings, such as large spikes in temperature that are not indicative of physiological changes (e.g., a temperature spike from a hot shower). In some implementations, the ring 104 may filter/reject temperature readings that may not be reliable due to other factors, such as excessive motion during exercise (e.g., as indicated by a motion sensor 245).

The ring 104 (e.g., communication module) may transmit the sampled and/or average temperature data to the user device 106 for storage and/or further processing. The user device 106 may transfer the sampled and/or average temperature data to the server 110 for storage and/or further processing.

Although the ring 104 is illustrated as including a single temperature sensor 240, the ring 104 may include multiple temperature sensors 240 in one or more locations, such as arranged along the inner housing 205-a near the user's finger. In some implementations, the temperature sensors 240 may be stand-alone temperature sensors 240. Additionally, or alternatively, one or more temperature sensors 240 may be included with other components (e.g., packaged with other components), such as with the accelerometer and/or processor.

The processing module 230-a may acquire and process data from multiple temperature sensors 240 in a similar manner described with respect to a single temperature sensor 240. For example, the processing module 230 may individually sample, average, and store temperature data from each of the multiple temperature sensors 240. In other examples, the processing module 230-a may sample the sensors at different rates and average/store different values for the different sensors. In some implementations, the processing module 230-a may be configured to determine a single temperature based on the average of two or more temperatures determined by two or more temperature sensors 240 in different locations on the finger.

The temperature sensors 240 on the ring 104 may acquire distal temperatures at the user's finger (e.g., any finger). For example, one or more temperature sensors 240 on the ring 104 may acquire a user's temperature from the underside of a finger or at a different location on the finger. In some implementations, the ring 104 may continuously acquire distal temperature (e.g., at a sampling rate). Although distal temperature measured by a ring 104 at the finger is described herein, other devices may measure temperature at the same/different locations. In some cases, the distal temperature measured at a user's finger may differ from the temperature measured at a user's wrist or other external body location. Additionally, the distal temperature measured at a user's finger (e.g., a “shell” temperature) may differ from the user's core temperature. As such, the ring 104 may provide a useful temperature signal that may not be acquired at other internal/external locations of the body. In some cases, continuous temperature measurement at the finger may capture temperature fluctuations (e.g., small or large fluctuations) that may not be evident in core temperature. For example, continuous temperature measurement at the finger may capture minute-to-minute or hour-to-hour temperature fluctuations that provide additional insight that may not be provided by other temperature measurements elsewhere in the body.

The ring 104 may include a PPG system 235. The PPG system 235 may include one or more optical transmitters that transmit light. The PPG system 235 may also include one or more optical receivers that receive light transmitted by the one or more optical transmitters. An optical receiver may generate a signal (hereinafter “PPG” signal) that indicates an amount of light received by the optical receiver. The optical transmitters may illuminate a region of the user's finger. The PPG signal generated by the PPG system 235 may indicate the perfusion of blood in the illuminated region. For example, the PPG signal may indicate blood volume changes in the illuminated region caused by a user's pulse pressure. The processing module 230-a may sample the PPG signal and determine a user's pulse waveform based on the PPG signal. The processing module 230-a may determine a variety of physiological parameters based on the user's pulse waveform, such as a user's respiratory rate, heart rate, HRV, oxygen saturation, and other circulatory parameters.

In some implementations, the PPG system 235 may be configured as a reflective PPG system 235 in which the optical receiver(s) receive transmitted light that is reflected through the region of the user's finger. In some implementations, the PPG system 235 may be configured as a transmissive PPG system 235 in which the optical transmitter(s) and optical receiver(s) are arranged opposite to one another, such that light is transmitted directly through a portion of the user's finger to the optical receiver(s).

The number and ratio of transmitters and receivers included in the PPG system 235 may vary. Example optical transmitters may include light-emitting diodes (LEDs). The optical transmitters may transmit light in the infrared spectrum and/or other spectrums. Example optical receivers may include, but are not limited to, photosensors, phototransistors, and photodiodes. The optical receivers may be configured to generate PPG signals in response to the wavelengths received from the optical transmitters. The location of the transmitters and receivers may vary. Additionally, a single device may include reflective and/or transmissive PPG systems 235.

The PPG system 235 illustrated in FIG. 2 may include a reflective PPG system 235 in some implementations. In these implementations, the PPG system 235 may include a centrally located optical receiver (e.g., at the bottom of the ring 104) and two optical transmitters located on each side of the optical receiver. In this implementation, the PPG system 235 (e.g., optical receiver) may generate the PPG signal based on light received from one or both of the optical transmitters. In other implementations, other placements, combinations, and/or configurations of one or more optical transmitters and/or optical receivers are contemplated.

The processing module 230-a may control one or both of the optical transmitters to transmit light while sampling the PPG signal generated by the optical receiver. In some implementations, the processing module 230-a may cause the optical transmitter with the stronger received signal to transmit light while sampling the PPG signal generated by the optical receiver. For example, the selected optical transmitter may continuously emit light while the PPG signal is sampled at a sampling rate (e.g., 250 Hz).

Sampling the PPG signal generated by the PPG system 235 may result in a pulse waveform that may be referred to as a “PPG.” The pulse waveform may indicate blood pressure vs time for multiple cardiac cycles. The pulse waveform may include peaks that indicate cardiac cycles. Additionally, the pulse waveform may include respiratory induced variations that may be used to determine respiration rate. The processing module 230-a may store the pulse waveform in memory 215 in some implementations. The processing module 230-a may process the pulse waveform as it is generated and/or from memory 215 to determine user physiological parameters described herein. The user physiological parameters may be a heart rate of the user (e.g., an interbeat interval (IBI)), a pulse wave amplitude, SpO2, and the like.

The processing module 230-a may determine the user's heart rate based on the pulse waveform. For example, the processing module 230-a may determine heart rate (e.g., in beats per minute) based on the time between peaks in the pulse waveform. The time between peaks may be referred to as an IBI. The processing module 230-a may store the determined heart rate values and IBI values in memory 215.

The processing module 230-a may determine HRV over time. For example, the processing module 230-a may determine HRV based on the variation in the IBIs. The processing module 230-a may store the HRV values over time in the memory 215. Moreover, the processing module 230-a may determine the user's respiratory rate over time. For example, the processing module 230-a may determine respiratory rate based on frequency modulation, amplitude modulation, or baseline modulation of the user's IBI values over a period of time. Respiratory rate may be calculated in breaths per minute or as another breathing rate (e.g., breaths per 30 seconds). The processing module 230-a may store user respiratory rate values over time in the memory 215.

The ring 104 may include one or more motion sensors 245, such as one or more accelerometers (e.g., 6-D accelerometers) and/or one or more gyroscopes (gyros). The motion sensors 245 may generate motion signals that indicate motion of the sensors. For example, the ring 104 may include one or more accelerometers that generate acceleration signals that indicate acceleration of the accelerometers. As another example, the ring 104 may include one or more gyro sensors that generate gyro signals that indicate angular motion (e.g., angular velocity) and/or changes in orientation. The motion sensors 245 may be included in one or more sensor packages. An example accelerometer/gyro sensor is a Bosch BM1160 inertial micro electro-mechanical system (MEMS) sensor that may measure angular rates and accelerations in three perpendicular axes.

The processing module 230-a may sample the motion signals at a sampling rate (e.g., 50 Hz) and determine the motion of the ring 104 based on the sampled motion signals. For example, the processing module 230-a may sample acceleration signals to determine acceleration of the ring 104. As another example, the processing module 230-a may sample a gyro signal to determine angular motion. In some implementations, the processing module 230-a may store motion data in memory 215. Motion data may include sampled motion data as well as motion data that is calculated based on the sampled motion signals (e.g., acceleration and angular values).

The ring 104 may store a variety of data described herein. For example, the ring 104 may store temperature data, such as raw sampled temperature data and calculated temperature data (e.g., average temperatures). As another example, the ring 104 may store PPG signal data, such as pulse waveforms and data calculated based on the pulse waveforms (e.g., heart rate values, IBI values, HRV values, and respiratory rate values). The ring 104 may also store motion data, such as sampled motion data that indicates linear and angular motion.

The ring 104, or other computing device, may calculate and store additional values based on the sampled/calculated physiological data. For example, the processing module 230 may calculate and store various metrics, such as sleep metrics (e.g., a Sleep Score), activity metrics, and readiness metrics. In some implementations, additional values/metrics may be referred to as “derived values.” The ring 104, or other computing/wearable device, may calculate a variety of values/metrics with respect to motion. Example derived values for motion data may include, but are not limited to, motion count values, regularity values, intensity values, metabolic equivalence of task values (METs), and orientation values. Motion counts, regularity values, intensity values, and METs may indicate an amount of user motion (e.g., velocity/acceleration) over time. Orientation values may indicate how the ring 104 is oriented on the user's finger and if the ring 104 is worn on the left hand or right hand.

In some implementations, motion counts and regularity values may be determined by counting a number of acceleration peaks within one or more periods of time (e.g., one or more 30 second to 1 minute periods). Intensity values may indicate a number of movements and the associated intensity (e.g., acceleration values) of the movements. The intensity values may be categorized as low, medium, and high, depending on associated threshold acceleration values. METs may be determined based on the intensity of movements during a period of time (e.g., 30 seconds), the regularity/irregularity of the movements, and the number of movements associated with the different intensities.

In some implementations, the processing module 230-a may compress the data stored in memory 215. For example, the processing module 230-a may delete sampled data after making calculations based on the sampled data. As another example, the processing module 230-a may average data over longer periods of time in order to reduce the number of stored values. In a specific example, if average temperatures for a user over one minute are stored in memory 215, the processing module 230-a may calculate average temperatures over a five minute time period for storage, and then subsequently erase the one minute average temperature data. The processing module 230-a may compress data based on a variety of factors, such as the total amount of used/available memory 215 and/or an elapsed time since the ring 104 last transmitted the data to the user device 106.

Although a user's physiological parameters may be measured by sensors included on a ring 104, other devices may measure a user's physiological parameters. For example, although a user's temperature may be measured by a temperature sensor 240 included in a ring 104, other devices may measure a user's temperature. In some examples, other wearable devices (e.g., wrist devices) may include sensors that measure user physiological parameters. Additionally, medical devices, such as external medical devices (e.g., wearable medical devices) and/or implantable medical devices, may measure a user's physiological parameters. One or more sensors on any type of computing device may be used to implement the techniques described herein.

The physiological measurements may be taken continuously throughout the day and/or night. In some implementations, the physiological measurements may be taken during portions of the day and/or portions of the night. In some implementations, the physiological measurements may be taken in response to determining that the user is in a specific state, such as an active state, resting state, and/or a sleeping state. For example, the ring 104 can make physiological measurements in a resting/sleep state in order to acquire cleaner physiological signals. In one example, the ring 104 or other device/system may detect when a user is resting and/or sleeping and acquire physiological parameters (e.g., temperature) for that detected state. The devices/systems may use the resting/sleep physiological data and/or other data when the user is in other states in order to implement the techniques of the present disclosure.

In some implementations, as described previously herein, the ring 104 may be configured to collect, store, and/or process data, and may transfer any of the data described herein to the user device 106 for storage and/or processing. In some aspects, the user device 106 includes a wearable application 250, an operating system (OS), a web browser application (e.g., web browser 280), one or more additional applications, and a GUI 275. The user device 106 may further include other modules and components, including sensors, audio devices, haptic feedback devices, and the like. The wearable application 250 may include an example of an application (e.g., “app”) that may be installed on the user device 106. The wearable application 250 may be configured to acquire data from the ring 104, store the acquired data, and process the acquired data as described herein. For example, the wearable application 250 may include a user interface (UI) module 255, an acquisition module 260, a processing module 230-b, a communication module 220-b, and a storage module (e.g., database 265) configured to store application data.

The various data processing operations described herein may be performed by the ring 104, the user device 106, the servers 110, or any combination thereof. For example, in some cases, data collected by the ring 104 may be pre-processed and transmitted to the user device 106. In this example, the user device 106 may perform some data processing operations on the received data, may transmit the data to the servers 110 for data processing, or both. For instance, in some cases, the user device 106 may perform processing operations that require relatively low processing power and/or operations that require a relatively low latency, whereas the user device 106 may transmit the data to the servers 110 for processing operations that require relatively high processing power and/or operations that may allow relatively higher latency.

In some aspects, the ring 104, user device 106, and server 110 of the system 200 may be configured to evaluate sleep patterns for a user. In particular, the respective components of the system 200 may be used to collect data from a user via the ring 104, and generate one or more scores (e.g., Sleep Score, Readiness Score) for the user based on the collected data. For example, as noted previously herein, the ring 104 of the system 200 may be worn by a user to collect data from the user, including temperature, heart rate, HRV, and the like. Data collected by the ring 104 may be used to determine when the user is asleep in order to evaluate the user's sleep for a given “sleep day.” In some aspects, scores may be calculated for the user for each respective sleep day, such that a first sleep day is associated with a first set of scores, and a second sleep day is associated with a second set of scores. Scores may be calculated for each respective sleep day based on data collected by the ring 104 during the respective sleep day. Scores may include, but are not limited to, Sleep Scores, Readiness Scores, and the like.

In some cases, “sleep days” may align with the traditional calendar days, such that a given sleep day runs from midnight to midnight of the respective calendar day. In other cases, sleep days may be offset relative to calendar days. For example, sleep days may run from 6:00 pm (18:00) of a calendar day until 6:00 pm (18:00) of the subsequent calendar day. In this example, 6:00 pm may serve as a “cut-off time,” where data collected from the user before 6:00 pm is counted for the current sleep day, and data collected from the user after 6:00 pm is counted for the subsequent sleep day. Due to the fact that most individuals sleep the most at night, offsetting sleep days relative to calendar days may enable the system 200 to evaluate sleep patterns for users in such a manner that is consistent with their sleep schedules. In some cases, users may be able to selectively adjust (e.g., via the GUI) a timing of sleep days relative to calendar days so that the sleep days are aligned with the duration of time in which the respective users typically sleep.

In some implementations, each overall score for a user for each respective day (e.g., Sleep Score, Readiness Score) may be determined/calculated based on one or more “contributors,” “factors,” or “contributing factors.” For example, a user's overall Sleep Score may be calculated based on a set of contributors, including: total sleep, efficiency, restfulness, REM sleep, deep sleep, latency, timing, or any combination thereof. The Sleep Score may include any quantity of contributors. The “total sleep” contributor may refer to the sum of all sleep periods of the sleep day. The “efficiency” contributor may reflect the percentage of time spent asleep compared to time spent awake while in bed, and may be calculated using the efficiency average of long sleep periods (e.g., primary sleep period) of the sleep day, weighted by a duration of each sleep period. The “restfulness” contributor may indicate how restful the user's sleep is, and may be calculated using the average of all sleep periods of the sleep day, weighted by a duration of each period. The restfulness contributor may be based on a “wake up count” (e.g., sum of all the wake-ups (when user wakes up) detected during different sleep periods), excessive movement, and a “got up count” (e.g., sum of all the got-ups (when user gets out of bed) detected during the different sleep periods).

The “REM sleep” contributor may refer to a sum total of REM sleep durations across all sleep periods of the sleep day including REM sleep. Similarly, the “deep sleep” contributor may refer to a sum total of deep sleep durations across all sleep periods of the sleep day including deep sleep. The “latency” contributor may signify how long (e.g., average, median, longest) the user takes to go to sleep, and may be calculated using the average of long sleep periods throughout the sleep day, weighted by a duration of each period and the number of such periods (e.g., consolidation of a given sleep stage or sleep stages may be its own contributor or weight other contributors). Lastly, the “timing” contributor may refer to a relative timing of sleep periods within the sleep day and/or calendar day, and may be calculated using the average of all sleep periods of the sleep day, weighted by a duration of each period.

By way of another example, a user's overall Readiness Score may be calculated based on a set of contributors, including: sleep, sleep balance, heart rate, HRV balance, recovery index, temperature, activity, activity balance, or any combination thereof. The Readiness Score may include any quantity of contributors. The “sleep” contributor may refer to the combined Sleep Score of all sleep periods within the sleep day. The “sleep balance” contributor may refer to a cumulative duration of all sleep periods within the sleep day. In particular, sleep balance may indicate to a user whether the sleep that the user has been getting over some duration of time (e.g., the past two weeks) is in balance with the user's needs. Typically, adults need 7-9 hours of sleep a night to stay healthy, alert, and to perform at their best both mentally and physically. However, it is normal to have an occasional night of bad sleep, so the sleep balance contributor takes into account long-term sleep patterns to determine whether each user's sleep needs are being met. The “resting heart rate” contributor may indicate a lowest heart rate from the longest sleep period of the sleep day (e.g., primary sleep period) and/or the lowest heart rate from naps occurring after the primary sleep period.

Continuing with reference to the “contributors” (e.g., factors, contributing factors) of the Readiness Score, the “HRV balance” contributor may indicate a highest HRV average from the primary sleep period and the naps happening after the primary sleep period. The HRV balance contributor may help users keep track of their recovery status by comparing their HRV trend over a first time period (e.g., two weeks) to an average HRV over some second, longer time period (e.g., three months). The “recovery index” contributor may be calculated based on the longest sleep period. Recovery index measures how long it takes for a user's resting heart rate to stabilize during the night. A sign of a very good recovery is that the user's resting heart rate stabilizes during the first half of the night, at least six hours before the user wakes up, leaving the body time to recover for the next day. The “body temperature” contributor may be calculated based on the longest sleep period (e.g., primary sleep period) or based on a nap happening after the longest sleep period if the user's highest temperature during the nap is at least 0.5° C. higher than the highest temperature during the longest period. In some aspects, the ring may measure a user's body temperature while the user is asleep, and the system 200 may display the user's average temperature relative to the user's baseline temperature. If a user's body temperature is outside of their normal range (e.g., clearly above or below 0.0), the body temperature contributor may be highlighted (e.g., go to a “Pay attention” state) or otherwise generate an alert for the user.

In some aspects, the system 200 may support techniques for triggering an SpO2, or oxygen saturation, measurement at one or more sensors (e.g., sensors of the PPG system 235) of a wearable device 104. The PPG measurements may include two separate optical channel PPG measurements to obtain an SpO2 measurement. For example, a user device 106 may select a triggering condition (e.g., based on implementing a learning model), which may be a physical state of the wearable device, a physiological state of the user, or both. In some cases, the wearable device 104 may record temperature measurements, the PPG measurements, motion measurements, pressure measurements, SpO2 measurements, or any combination thereof using the temperature sensors 240, the PPG system 235, and any other sensors on of the wearable device 104. A user device 106, or another device with access to the data, may use the temperature data, the PPG data, the motion data, the pressure data, or any combination thereof as well as application data or other physiological data to determine the triggering condition.

In some aspects, the system 200 may support techniques for determining a breathing quality metric associated with a user. For example, the system 200 may be triggered to perform one or more physiological measurements (e.g., SpO2 measurements, other PPG measurements, temperature measurements, motion measurements, audio measurements) using one or more triggering techniques described herein. The system 200 may input some or all of the physiological measurements, as application data or other physiological data, into a neural network, which may output a breathing quality metric such as a probability that the user experiences apnea, hypopnea, oxygen desaturation, or RERA. The system may determine an index (e.g., a BDI, an AHI, an ODI) associated with the breathing quality, and may display the index via the GUI (e.g., a GIU of a user device 106).

FIG. 3 illustrates an example of a wearable device diagram 300 that supports oxygen saturation measurement and reporting in accordance with aspects of the present disclosure. The wearable device diagram 300 may illustrate triggering an oxygen saturation, or SpO2, measurement at a wearable device 104-d using a user device 106-c, which may be examples of wearable devices 104 and user devices 106, respectively, as described with reference to FIGS. 1 and 2. A user may place the wearable device 104-d at different localities of an anatomical feature 305 (e.g., a finger), which may satisfy a condition to trigger the measurements. Although the wearable device 104-d is illustrated as a ring in FIG. 3, the wearable device 104-d may be any example of a wearable device (e.g., a watch, a necklace, and the like).

The anatomical feature 305 may include one or more elements, such as capillaries 310 and arteries 315 (e.g., veins of a user) through which blood may flow. The capillaries 310 and the arteries 315 may have muscle tissue in the vein walls, which may impact the amount of light that may penetrate the capillaries 310 and the arteries 315. For example, the arteries 315 may have thicker muscle tissue than the arteries 315, such that the light may penetrate the walls of the capillaries 310 more effectively than the walls of the arteries 315. Additionally, or alternatively, the capillaries 310 may be a shorter distance below the surface of the anatomical feature 305 than the arteries 315, such that one or more wearable device sensors, which may be one or more wearable device sensors as described with reference to FIG. 2, may collect more accurate measurements from the capillaries 310. For example, the arteries 315, which may represent human arteries, may be located 3 millimeters (mm) below the surface of the anatomical feature 305. The arteries 315 may have a threshold diameter, such as 1.2 mm. The capillaries 310, which may be representative of human capillaries, may be closer to the surface of the anatomical feature 305 than the arteries 315 (e.g., 10 microns below the surface). The capillaries 310 may branch out from the arteries 315 to create layers of human veins. Thus, in some examples, a wearable device sensor may test different penetration depths to collect measurements (e.g., SpO2 measurements).

In addition to the capillaries 310 and the arteries 315, the anatomical feature 305 may include one or more of human bone, human ligaments, human nails, human muscle, or any other aspects of a human appendage (e.g., finger), which may have optical, thermal, or mechanical properties that impact a measurement from one or more wearable device sensor of the wearable device 104-d. That is, the elements may have different light transmission and scatter and absorption properties, such as scatter and absorption properties related to a signal from pulsating blood-containing tissue in a PPG measurement (e.g., SpO2 measurement). In some examples, a set of wavelengths may penetrate the surface of the anatomical feature 305, such as 940 nanometers (nm) for human skin. The wearable device 104-d may perform one or more measurements, such as heart rate and oxygen saturation (e.g., SpO2) measurements.

In some examples, one or more users, such as users 102 as described with reference to FIG. 1, may use a wearable device 104-d to collect one or more health metrics, such as heart rate, body temperature, oxygen saturation, movement, etc. Each user may have unique attributes, such that an average health metric for each user may vary. For example, a user may have a different average heart rate, body temperature, or the like when compared with another user. Similarly, different localities of the anatomical feature 305 of a user may have unique attributes (e.g., due to the variation in elements at the different localities). Further, the environment of the user may affect the data collected by the wearable device 104-d. For example, the time of day, the outdoor temperature, the activities of the user, or any other external factor may affect the measurements of the wearable device 104-d. Due to the variations, it may be difficult to use the data from each user and different localities of a user to detect health related trends. For example, if a user has a naturally high or low heart rate, the wearable device 104-d may misdiagnose the high heart rate as a trend towards an illness and may not detect a high heart rate trend towards an illness for the low heart rate user, instead detecting a heart rate within a “normal” range.

Thus, as described herein, a user device 106-c may trigger an SpO2 measurement at a wearable device 104-d based on a triggering condition being satisfied. The triggering condition may be based on a physical state of the wearable device 104-d, a physiological state of the user, or both. For example, the user device 106-c may determine that a locality 320 of the anatomical feature 305 of the user provides a relatively accurate SpO2 measure when compared with other localities (e.g., due to the vein structure at the locality 320, a light interference condition at the locality 320, a pressure of the wearable device 104-d on the anatomical feature 305, or the like). The locality 320 may be towards a fingertip if the anatomical feature 305 is a finger, due to the fingertip having multiple capillaries where tissue may be similar (e.g., predictable for sensor measurements). The triggering condition may be that a pressure between the anatomical feature 305 and the sensors of the wearable device 104-d meets or exceeds a pressure for relatively accurate SpO2 measurements (e.g., the wearable device 104-d is pressed against the skin of the user). In some cases, the triggering condition may be that an orientation of the wearable device 104-d (e.g., a rotation of the wearable device 104-d around the anatomical feature 305) provides for the sensors of the wearable device 104-d to collect relatively accurate SpO2 measurements. The user device 106-c may compare a series of SpO2 measurements at different positions (e.g., a fingertip versus a base of the finger), orientations, and pressures to determine a locality 320 with relatively accurate SpO2 measurements.

Additionally, or alternatively, the user device 106-c may identify that a physiological state of the user, such as a heart rate, a respiratory rate, a sound characteristic, or any combination thereof, satisfies a threshold for triggering the SpO2 measurement. The threshold may be preconfigured, or otherwise defined, at the user device 106-c. For example, if the rate of change of a heart rate of a user exceeds a threshold rate of change, the user device 106-c may trigger an SpO2 measurement at the wearable device 104-d. Similarly, if the rate of change of a respiratory rate of the user exceeds a threshold rate of change, the user device 106-c may trigger an SpO2 measurement at the wearable device 104-d, which is described in further detail with respect to FIG. 4. The user device 106-c may analyze the SpO2 measurements to determine one or more heart rate disturbances, breathing disturbances, or the like. For example, the user device 106-c may generate a timeline with the triggered SpO2 measurements. The user device 106-c may display the timeline to a user via a GUI of the user device 106-c.

In some examples, the user device 106-c may determine the condition for triggering the SpO2 measurement based on one or more relationships between sensor data from the wearable device 104-d, application data, physiological data from the wearable device 104-d, or any combination hereof. For example, the user device 106-c may collect application data from one or more applications, such as a location of the user, an activity status of the user, or the like. The user device 106-c may use the application data in conjunction with sensor data and physiological data reported from the wearable device 104-d to select a condition for triggering the SpO2 measurement. For example, the application data may indicate that the user is exercising, thus if the user device 106-c receives irregular heart rate measurements or respiratory rate measurements, the user device 106-c may determine not to trigger the SpO2 measurements. However, if the application data indicates that the user is stationary (e.g., not exercising) and the user device 106-c receives irregular heart rate measurements or respiratory rate measurements, the user device 106-c may trigger the SpO2 measurements.

In some cases, at 325, the user device 106-c may select the condition for triggering an SpO2 measurement report 330, an SpO2 measurement cycle, or both. For example, the user device 106-c may select a position, orientation, pressure, physiological data from the wearable device 104-d, or the like to trigger the SpO2 measurement report 330 based on the relationship between sensor data from the wearable device 104-d, application data, physiological data from the wearable device 104-d, or any combination hereof. The user device 106-c may select the condition to trigger the SpO2 measurement report 330 based on implementing a learning model at the user device 106-c. The learning model may map previous sets of SpO2 measurements for the user, an SpO2 measurement accuracy for each of the previous measurements, and a set of conditions for the previous oxygen saturation measurements.

In some examples, the learning model may be a machine learning model (e.g., a neural network). For example, the user device 106-c may implement a machine learning model to predict a triggering condition for the SpO2 measurement report. In some examples, the machine learning model may be an example of a deep learning machine learning model, where a deep learning machine learning model may include multiple layers of operations between input and output. For example, the machine learning model may represent a convolution neural network (CNN) model, a recurrent neural network (RNN) model, a generative adversarial network (GAN) model, or any other deep learning or other neural network model. In some examples, the machine learning model may represent a subset of RNN models, such as a long short-term memory (LSTM) model, where an LSTM model may involve learning and memorizing long-term dependencies over time to make predictions based on time series data. For example, the machine learning model may include an LSTM cell with a time-series input, and may transfer outputs from the LSTM cell into additional instances of the cell over time for selectively updating machine learning model values to make predictions. In some examples, the machine learning model may predict whether a selected SpO2 measurement triggering condition may remain preferred compared to a last triggering condition based on historical measurements. For example, the machine learning model may predict whether or not an orientation of the wearable device 104-d with a current highest accuracy for SpO2 measurements may have the highest accuracy for SpO2 measurements at a next triggering condition.

In some examples, the user device 106-c may train a machine learning model using a learning approach. For example, the user device 106-c may train a machine learning model using supervised, semi-supervised, or unsupervised learning. Supervised learning may involve machine learning model training based on labeled training data, which may include example input-output pairs, whereas unsupervised learning may involve machine learning model training based on unlabeled training data, including data without example input-output pairs. Semi-supervised learning may involve a small amount of labeled training data and a large amount of unlabeled training data. In some cases, the machine learning model may use supervised learning for triggering condition prediction.

In some examples, the user device 106-c may communicate with the wearable device 104-d via a signaling link 335-a. Similarly, the wearable device 104-d may communicate with the user device 106-c via a signaling link 335-b. For example, the user device 106-c may transmit a control signal 340 to the wearable device 104-d indicating for the wearable device 104-d to perform one or more SpO2 measurements. The user device 106-c may transmit the control signal 340 based on the conditions to trigger the SpO2 measurements being met. In some examples, the control signal may indicate for the wearable device 104-d to perform the SpO2 measurement in accordance with an SpO2 measurement cycle. The cycle may define a number of measurements for the wearable device 104-d to perform as well as a periodicity of the measurements. In some examples, the periodicity measurement cycle may be preconfigured at the user device 106-c and the wearable device 104-d. Additionally, or alternatively, the user device 106-c may select an SpO2 measurement cycle for the wearable device 104-d, such as based on a learning model.

The wearable device 104-d may perform one or more SpO2 measurements based on receiving the control signal 340. For example, the wearable device 104-d may update an SpO2 measurement cycle to accommodate the triggered SpO2 measurements. The wearable device 104-d may transmit the SpO2 measurements to the user device 106-c in the SpO2 measurement report 330. In some examples, the user device 106-c may display one or more messages at a GUI of the user device 106-c after receiving the SpO2 measurement report 330. For example, the user device 106-c may display an average SpO2 measurement value 345, such as 95.2%, for the user. The user device 106-c may compute the average from the SpO2 measurement report. Additionally, or alternatively, the user device 106-c may display one or more messages based on analyzing the SpO2 measurement report, such as a breathing regularity message 350, which is described in further detail with respect to FIG. 4.

In some examples, the wearable device 104-d may implement one or more techniques for reducing power consumption and extending battery life. For example, the wearable device 104-d may take periodic SpO2 measurements (e.g., while the user is sleeping), and may transmit the measured value and/or some analysis of the measured value to the user device 106-c (e.g., for display via a GUI). In some examples, rather than sending an SpO2 measurement to the user device 106-c after every measurement, the wearable device 104-d may evaluate whether a measured value is within some configurable threshold (e.g., 3-5%) of one or more previously measured values, and may send the measured value when there is a change in measurement above the configurable threshold. In this way, the wearable device 104-d may not need to send the measured value each time a measurement is taken, which may result in reduced power consumption at the wearable device 104-d (e.g., by reducing processing and activating antenna elements and chips associated with wireless communication).

In some aspects, one or more devices of the wearable device diagram 300 may support detecting breathing disturbance in users. For example, the one or more devices may use physiological measurements (e.g., SpO2 measurements, other PPG measurements, temperature measurements, motion measurements, audio measurements) collected via the wearable device 104-d, the user device 106-c, and/or a charger of the wearable device to determine if a user experiences sleep apnea, sleep hypopnea, breathing disturbance, oxygen saturation, RERA, and the like.

Breathing disturbance events (e.g., sleep apnea, sleep hypopnea, breathing disturbance, oxygen desaturation, RERA) may result in one or more physiological changes in a user. For example, a breathing disturbance event may cause features such as a SpO2 of the user to decrease, a body temperature (e.g., skin temperature) of the user to decrease, a pulse wave amplitude (e.g., a volume of blood in the tissue of the user) to decrease, a heart rate (e.g., IBI) of the user to increase and/or decrease, an intensity of motion of the user to increase, and an ambient noise level to increase (e.g., due to snoring). In some examples, the pulse wave amplitude of the user may decrease as blood vessels of the user constrict due to a decrease in oxygen (e.g., SpO2). The heart rate of the user may decrease at a beginning of an apnea-related event and increase at an end of an apnea-related event (e.g., due to apnea-related arousal).

Accordingly, the user device 106-c may use any combination of features described herein to determine if a user is experiencing a breathing disturbance event (e.g., upon being triggered to perform physiological measurements, as described herein). For example, the user device 106-c may trigger the wearable device 104-d to acquire physiological data (e.g., via SpO2 measurements, other PPG measurements such as IBI and pulse wave amplitude, temperature measurements, motion measurements, and/or audio measurements) upon detecting that the user is asleep (e.g., or that the user is in a certain stage of sleep). The user device 106-c may input some or all of the physiological data into a neural network, which may enable the user device 106-c to determine if the user experienced a breathing disturbance event. As described herein, the physiological data may be a time series of physiological data collected during the sleep period of the user. Each input parameter (e.g., each type of measurement) may be associated with a same or different sampling frequency and a uniform or irregular sampling rate.

The neural network may be trained on a training physiological dataset. The training physiological dataset may include physiological data acquired from one or more users and may include ground-truth labels that are indicative of the one or more users experiencing or not experiencing a breathing disturbance event. For example, a ground-truth label of the training physiological dataset may include a scoring (e.g., a human-expert scoring) of whether a physiological measurement is indicative of a breathing disturbance event. The ground-truth labels may therefore indicate whether the physiological dataset is indicative of breathing disturbance events (e.g., apnea, hypopnea, oxygen desaturation, RERA) throughout a sleep period (e.g., a night), as measured using polysomnography.

The neural network may include one or more sequential blocks with one or more hidden layers (e.g., convolutional or fully-connected layers). The neural network may further include one or more parameters or weights. By training the neural network on the training physiological dataset, the user device 106-c may determine values of the one or more parameters. For example, the user device 106-c may iteratively learn to decrease (e.g., minimize) an error between predicted labels (e.g., an output of the neural network using the training physiological dataset) and the ground-truth labels. Thus, training the neural network may increase an accuracy of the neural network in determining whether a set of physiological data is indicative of a breathing disturbance event.

The neural network may be one of a set of neural networks. For example, the user device 106-c may train a set of neural networks, and each neural network of the set of neural networks may be associated with a different combination of input parameters (e.g., different combinations of the acquired physiological data). In some examples, the user device 106-f may select one of the set of neural networks based on an availability and/or a quality of the acquired physiological data.

The user device 106-c may accordingly input some or all of the acquired physiological data (e.g., the time series associated with each input parameter) into the neural network. The neural network may output a probability time series indicating a probability that the user experienced a breathing disturbance event during one or more time periods (e.g., timestamps) during the sleep period of the user. An output time resolution associated with the neural network may be one or more seconds, minutes, hours, and so on. In some examples, the neural network may output a single probability (e.g., a probability of whether the user experienced a breathing disturbance event at any point during the sleep period).

The user device 106-c may binarize the probability time series. For example, the user device 106-c may determine, for each timestamp, whether the user experienced a breathing disturbance event. The user device 106-c may binarize the probability time series by comparing the probability of each time stamp to a threshold. For example, if the probability associated with a timestamp is greater than a threshold, the user device 106-c may determine that the user experienced a breathing disturbance event during the timestamp. If the probability associated with a timestamp is less than a threshold, the user device 106-c may determine that the user did not experience a breathing disturbance event during the timestamp. The probability time series or the binarized probability time series may be referred to herein as a breathing disturbance metric.

In some examples, the user device 106-c may generate information related to the breathing disturbance metric, such as an index (e.g., an AHI, a BDI, an ODI, an HB) or a timeline of breathing disturbance during the sleep period. In some aspects, the AHI may represent an average quantity of apneas (e.g., complete or almost complete cessation of airflow) and hypopneas (e.g., partial reduction in airflow) experienced by the user per hour of sleep. Mild apnea may correspond to an AHI between 5 and 15, moderate apnea may correspond to an AHI between 15 and 30, and severe apnea may correspond to an AHI over 30. The ODI may represent an average quantity of times per hour that the user's SpO2 decreased by a certain percentage (e.g., from a baseline SpO2 of the user), such as 3 or 4 percent. The HB may represent a total area under an oxygen saturation curve (e.g., relative to a baseline oxygen saturation), which may provide a metric of a severity of oxygen desaturation in individuals with obstructive sleep apnea. A higher index (e.g., AHI, ODI, or BDI, or HB) may indicate that the user experienced a relatively greater quantity of breathing disturbance events than a lower index. The user device 106-c may cause a GUI to display the index and/or the timeline via a GUI of the user device 106-c, as described herein with reference to FIG. 4.

In some examples, the user device 106-c may determine (e.g., calculate) a breathing quality metric (e.g., a breathing Score). The breathing quality metric may be an inverse of the index, such that a relatively higher breathing quality metric is indicative of relatively fewer breathing disturbance events, a relatively higher average SpO2, a relatively higher pulse wave amplitude, and so on than a relatively lower breathing quality metric. The user device 106-c may display the breathing quality metric via the GUI.

In some examples, the user device 106-c may perform one or more actions in response to the breathing disturbance metric. For example, if the breathing disturbance metric satisfies a threshold, the user device 106-c may trigger an alarm (e.g., an auditory alarm, a visual notification, a vibration) to alert the user (e.g., or a caretaker of the user, such as a healthcare worker) that the user may be experiencing a breathing disturbance event, may have an SpO2 or heart rate that is below a threshold, and so on. In some examples, an audible alarm triggered by the breathing disturbance metric exceeding the threshold may have a volume that is above a threshold volume (e.g., a volume loud enough to wake the user from sleep or to notify a caregiver). In some examples, the alarm may be produced from the user device 106-c, from a charger of the wearable device 104-d, from the wearable device 104-d, or from another connected device.

In some examples, the process of acquiring physiological data and generating the breathing disturbance metric and/or index may be repeated one or more times (e.g., during one or more sleep periods or nights that the user wears the wearable device 104-d). Accordingly, the user may acquire continuous and extended estimates of breathing disturbance severity over time. The user device 106-c may average breathing disturbance metric data collected over multiple sleep periods of the user (e.g., multiple days) to provide a more robust assessment of sleep apnea severity as compared to data collected during a single sleep period. Such longitudinal monitoring of breathing disturbance may allow the user device 106-c to assess an effectiveness of interventions or lifestyle changes (e.g., changes in eating or drinking habits, changes in sleep habits, medication) on the breathing disturbance of the user. The user may therefore use personalized and proactive management strategies to decrease a risk of breathing disturbance events.

FIG. 4 illustrates an example of a GUI 400 that supports oxygen saturation measurement and reporting in accordance with aspects of the present disclosure. The GUI 400 may implement, or be implemented by, aspects of the system 100, the system 200, the wearable device diagram 300, or any combination thereof. For example, the GUI 400 may illustrate examples of a user device 106-d reporting an oxygen saturation value or analysis of SpO2 measurements to a user, where the user device 106-d may be an example of a user device 106 as described with reference to FIG. 1.

The user device 106-d may indicate one or more messages to a user via the GUI 400 of the user device 106-d. The messages may inform the user of one or more measured results or analysis of measurements. For example, the user device 106-d may instruct a wearable device to perform one or more physiological measurements (e.g., SpO2 measurements, pulse wave amplitude measurements, IBI measurements, skin temperature measurements, motion measurements, audio measurements), as described with reference to FIGS. 1, 2, and 3. The user device 106-d may perform one or more calculations to analyze the physiological (e.g., SpO2) measurements. For example, the user device 106-d may time-average the physiological (e.g., SpO2) measurements, and may display the time-averaged physiological (e.g., SpO2) measurements to the user via the GUI 400. In some other examples, the user device 106-d may use the physiological (e.g., SpO2) measurements to identify one or more breathing disturbance events 410 of the user. For example, if the rate of change of a user's SpO2 value is significant (e.g., above a threshold value) between measurements, the user's SpO2 decreases relative to a baseline, the user's IBI increases, the user's pulse wave amplitude decreases, the user's body temperature decreases, an intensity of motion of the user increases, or an ambient sound level increases, the user device 106-d may flag those features as possible breathing disturbances 410 (e.g., via a neural network, as described herein). The user device 106-d may display the breathing disturbances 410 to the user via a breathing regularity message 415. Breathing disturbances 410 may be examples of apnea events, hypopnea events, RERA events, and/or oxygen desaturation events.

In some examples, the user device 106-d may categorize (e.g., classify) the breathing disturbances into one or more categories based on breathing disturbance thresholds for each category. For example, the categories may include a very regular breathing pattern 420, a mostly regular breathing pattern 425, an irregular breathing pattern 430, and a very irregular breathing pattern 435, among others. In some examples, the threshold number of breathing disturbances 410 for the very regular breathing pattern 420 may be close to, or at, zero; the threshold number of breathing disturbances 410 for the mostly regular breathing pattern 425 may be between zero and 5; the threshold number of breathing disturbances 410 for the irregular breathing pattern 430 may be between 5 and 10; and the threshold number of breathing disturbances for the very irregular breathing pattern 435 may be over 10. In some other examples, the breathing disturbance 410 thresholds may be tailored to individual users based on individual physiological data and history, and/or based on demographic data from similarly situated users. The breathing disturbance thresholds may be based on associated AHI, BDI, or HB thresholds, as described herein.

The breathing regularity message 415 may include a verbal assessment of a user's average breathing regularity over a period of time, such as when the user is asleep. The breathing regularity message 415 may additionally, or alternatively, include an explanation of the verbal assessment as well as a timeline that shows when suspected breathing disturbances 410 may have occurred and how severe the disturbances were. For example, for the very regular breathing pattern 420, the breathing regularity message 415 may recite “No significant variations in your blood oxygen levels were detected. This can indicate that you experienced no breathing disturbances in your sleep.” Similarly, for the mostly regular breathing pattern 425, the breathing regularity message 415 may recite “Some variations in your blood oxygen levels were detected. This can indicate that you experienced occasional breathing disturbances in your sleep.”

For the irregular breathing pattern 430, the breathing regularity message 415 may recite “Several variations in your blood oxygen levels were detected. This can indicate that you experienced many breathing disturbances during your sleep.” Finally, for the very irregular breathing pattern 435, the breathing regularity message 415 may recite “Frequent variations in your blood oxygen levels were detected. This can indicate that you experienced a great number of breathing disturbances during your sleep.” The severity of the breathing disturbances 410 may be shown as a length of a vertical line in the timeline, a color of a vertical line in the timeline, or the like. In some examples, the severity of the breathing disturbances 410 may be categorized into multiple categories, such as a mild disturbance shown as a short, green line, a moderate disturbance shown as a mid-length, yellow line, or a severe disturbance shown as a long, red line. The user device 106-d may sort the breathing disturbance events into the three categories based on thresholds for each respective category.

The breathing regularity message 415, or section, on the GUI 400 may be placed between one or more other data analysis charts, such as a hypnogram and a resting heart rate graph. The user, the user device 106-d, or both may analyze whether possible breathing disturbances 410 occur within a sleep stage, or if the breathing disturbances 410 may have some effects on a resting heart rate. The breathing regularity message 415 may include a description text block, which may tell the user that the breathing regularity assessment is based on the blood oxygen variations detected during their sleep, and how blood oxygen variation and breathing regularity are connected. In some examples, the breathing regularity assessment may be available for one or more sleep periods of the day (e.g., a longest sleep period of the day).

The breathing regularity message 415 may include a timeline visualization, which may show when suspected breathing disturbances 410 occur and the severity, or intensity, of the possible disturbances. The suspected breathing disturbances timeline may be horizontally positioned so that the disturbance indicators are aligned with the hypnogram, resting heart rate, and HRV graphs. This way, the user may analyze whether possible breathing disturbances occur within a sleep stage, or if they have some effects on their resting heart rate. Additionally, or alternatively, the GUI 400 may display an analysis of the breathing disturbances, which may include an alert of a possible health condition, a recommendation to prevent future breathing disturbances, or the like. For example, the user device 106-d may analyze the breathing disturbances over time to diagnose an illness, a health condition, or both (e.g., apnea, hypopnea, and the like). The GUI 400 of the user device 106-d may indicate to the user the diagnosis. The suspected breathing disturbances timeline may have a similar visual look to other display messages at the GUI 400 (e.g., a nighttime movement graph). In some examples, the suspected breathing disturbances timeline highlights the bedtime start and end times for the user.

In some examples, if the process of acquiring physiological data and generating the breathing disturbance metric and/or index is repeated one or more times (e.g., during one or more sleep periods or nights that the user wears the wearable device 104-d), the wearable device may assess an effectiveness of interventions or lifestyle changes (e.g., changes in eating or drinking habits, changes in sleep habits, medication) on the breathing disturbance of the user. The GUI 400 may accordingly display one or more recommendations to decrease a quantity of breathing disturbance events experienced by the user. As an illustrative example, the GUI 400 may display a message that states “You are more likely to experience breathing disturbances when you drink alcohol before bed. We recommend decreasing your alcohol intake to improve your sleep quality.” The user may therefore use personalized and proactive management strategies to decrease a risk of breathing disturbance events. In some examples, the GUI may include a clinician-facing feature, such that a clinician of the user may analyze breathing disturbance data (e.g., the timeline, a BDI, an AHI, an HB, and the like) and provide the recommendation to the user.

In some examples, a user may opt-in to the SpO2 measurements and/or breathing disturbance detection. For example, the user may interact with the GUI 400 to enable the SpO2 measurements and/or breathing disturbance detection indefinitely, or for a period of time (e.g., a number of days). The GUI 400 may display a toggle that enables and disables the SpO2 measurements and/or breathing disturbance detection.

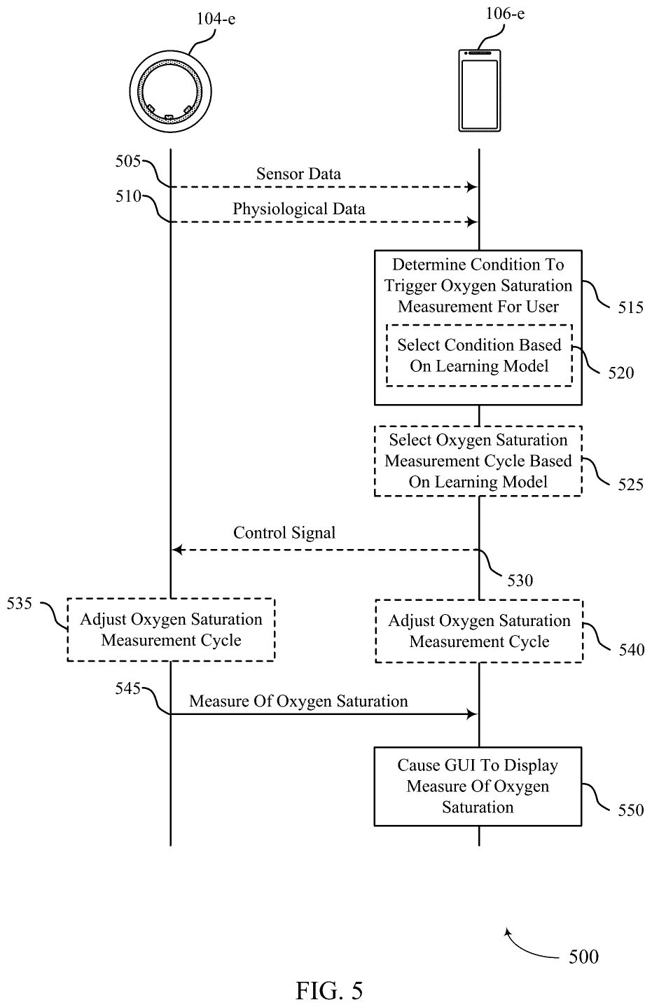

FIG. 5 illustrates an example of a process flow 500 that supports oxygen saturation measurement and reporting in accordance with aspects of the present disclosure. In some examples, the process flow 500 may implement aspects of the system 100, the system 200, the wearable device diagram 300, the GUI 400, or any combination thereof. The process flow 500 may illustrate an example of a user device 106-e triggering an SpO2 measurement at a wearable device 104-e based on a triggering condition. The user device 106-e and the wearable device 104-e may be examples of a user device 106 and a wearable device 104, respectively, as described with reference to FIGS. 1 and 2. Alternative examples of the following may be implemented, where some processes are performed in a different order than described or are not performed. In some cases, processes may include additional features not mentioned below, or further processes may be added.

At 505, the wearable device 104-e may transmit sensor data to the user device 106-e. For example, the sensor data may indicate a position value of the wearable device 104-e relative to an anatomical feature of the user (e.g., on a finger of the user if the wearable device 104-e is a ring). The position value may indicate a physical locality of the wearable device 104-e on the anatomical feature, which may be included in a physical state of the wearable device 104-e. The user device 106-e may determine that the position value satisfies a threshold position value for a threshold duration, where the threshold position value and the threshold duration may be defined at the user device 106-e, may be determined through testing, or the like.

Additionally, or alternatively, the wearable device 104-e may transmit an orientation value of the wearable device 104-e relative to the anatomical feature (e.g., finger) of the user, where the orientation value may be a degree of rotation around the anatomical feature. The orientation value may be included in the physical state of the wearable device 104-e. The user device 106-e may determine that the orientation value satisfies a threshold orientation value for a threshold duration, where the threshold orientation value and the threshold duration may be defined at the user device 106-e, may be determined through testing, or the like.

In some examples, the wearable device 104-e may transmit a pressure value to the user device 106-e, where the pressure value includes a pressure between the wearable device 104-e and the anatomical feature (e.g., the skin of a finger of the user). The pressure value may indicate a force of the wearable device 104-e on the anatomical feature, which may be included in the physical state of the wearable device 104-e. The user device 106-e may determine that the pressure value between the wearable device 104-e and the anatomical feature satisfies a threshold pressure value for a threshold duration, where the threshold pressure value and the threshold duration may be defined at the user device 106-e, may be determined through testing, or the like. In some examples, the wearable device 104-e may transmit a sound characteristic for the user to the user device 106-e. The user device 106-e may determine that the sound characteristic satisfies a threshold value.

At 510, the user device 106-e may receive physiological data from the wearable device 104-e and may determine that one or more physiological parameters of the physiological data satisfy a threshold. For example, the user device 106-e may receive heart rate data for the user from the user device 106-e. The heart rate data may be included in a physiological state of the user. In some cases, the user device 106-e may detect an abnormal heart rate of the user using the heart rate data, where the abnormal heart rate differs from a range of heart rate values of the user. Additionally, or alternatively, the user device 106-e may receive an indication of a sleep state of the user based on the physiological data, including one or more breathing rate measurements. In some examples, the user device 106-e may receive application data from one or more applications executable on the user device 106-e. For example, the applications may include a lifestyle application, a social media application, a utility application, an information outlet application, or any combination thereof. The application data may be indicative of an activity the user is engaged in or a location of the user, or any combination thereof.

At 515, the user device 106-e may determine a condition to trigger one or more oxygen saturation measurements (e.g., SpO2 measurements) at the wearable device 104-c. For example, the user device 106-e may determine the threshold position value and duration are satisfied, the threshold orientation value and duration are satisfied, the threshold pressure value and duration are satisfied, the threshold sound characteristic is satisfied, the physiological parameters of the physiological data satisfy the threshold (e.g., detecting an abnormal heart rate, a sleep state of the user, or the like), or any combination thereof, which may trigger the one or more oxygen saturation measurements. Additionally, or alternatively, the user device 106-e may determine the condition to trigger the SpO2 measurement based on the activity the user is engaged in or the location of the user according to the application data. In some examples, the condition to trigger the SpO2 measurement for a user may be based on a physical state of the wearable device, a physiological state of the user, or both.

The user device 106-e may determine the condition based on one or more relationships between the sensor data from the wearable device, the application data, the physiological data from the wearable device, or any combination thereof. For example, at 520, the user device 106-e may select the condition to trigger the SpO2 measurement for the user based on implementing a learning model (e.g., a neural network). The learning model may include a mapping between a set of previous SpO2 measurements for the user, a respective SpO2 measurement accuracy for each of the previous SpO2 measurements, and a set of conditions that triggered the previous SpO2 measurements. In some examples, the user device 106-e may determine a duration between a timestamp of a previously performed SpO2 measurement operation at the user device 106-e, the wearable device 104-e, or both, and a timestamp for determining that the condition to enable the SpO2 measurement satisfies (e.g., is above or below) a threshold. The duration satisfying the threshold may be the triggering condition for the SpO2 measurements.

At 525, the user device 106-e may select an SpO2 measurement cycle from a set of SpO2 measurement cycles using a learning model, which may be the same as or different from the learning model at 520. For example, the learning model may include mapping between one or more conditions of a set of conditions and one or more SpO2 measurement cycles.

At 530, the user device 106-e may transmit a control signal to the wearable device 104-e indicating for the wearable device 104-e to perform an SpO2 measurement for the user in accordance with an oxygen saturation measurement cycle based on the determined condition. For example, the control signal may indicate for the wearable device 104-e to perform the SpO2 measurement using the selected oxygen saturation measurement cycle, which may include a number of SpO2 measurements to perform and a periodicity for the wearable device 104-e to perform the SpO2 measurements, transmit the SpO2 measurements, or both. In some examples, the user device 106-e may receive an input to trigger the SpO2 measurement for the user based on a user input or a setting executable via an application running on the user device 106-e.

At 535 and 540, the wearable device 104-e and the user device 106-e may adjust an oxygen saturation measurement cycle, respectively. For example, the wearable device 104-e and the user device 106-e may adjust the SpO2 measurement cycle from a default SpO2 measurement cycle based on the condition.

At 545, the wearable device 104-e may perform one or more SpO2 measurements (e.g., in accordance with the control signal) based on the condition that triggers the SpO2 measurements. The wearable device 104-e may transmit the SpO2 measurements (e.g., SpO2 values) to the user device 106-e. In some examples, the user device 106-e may use the SpO2 measurements to determine one or more breathing disturbances for the user during a duration of a sleep state of the user. For example, the user device 106-e may trigger the SpO2 measurements for the user during the duration.

At 550, the user device 106-e may cause a GUI of the user device 106-e to display an indication of the SpO2 measurements for the user, such as an average SpO2 value, an analysis of the SpO2 measurements, or both. For example, the user device 106-e may cause the GUI to display one or more breathing disturbances of the user. Additionally, or alternatively, the user device 106-e may cause the GUI to display an average breathing regularity during a duration of a sleep state of the user, a description of the average breathing regularity, a respective timestamp of the one or more breathing disturbances, a magnitude of the one or more breathing disturbances, or any combination thereof. In some other examples, the user device 106-e may analyze the SpO2 measurements to detect a health condition, such as an illness, of a user. The GUI of the user device 106-e may alert the user of the detected health condition by displaying a message to the user. The user may act on the message by acknowledging the indication and scheduling an appointment with a healthcare professional.

In some examples, the wearable device 104-e may be an example of a wearable ring device, a wearable bracelet or watch device, a wearable necklace device, a wearable earring device, or any other form of a wearable device.

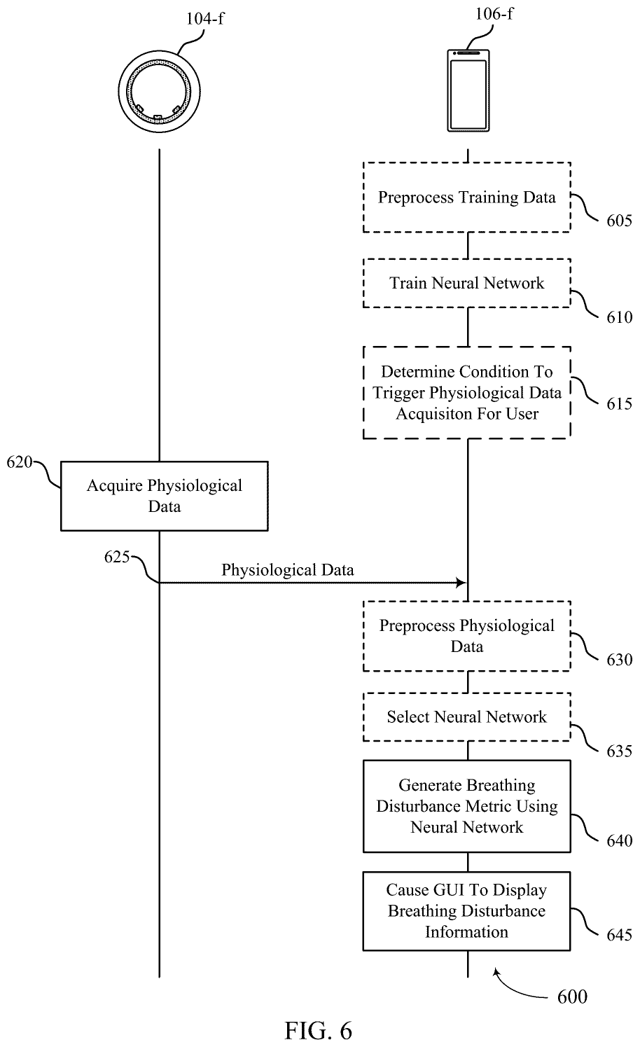

FIG. 6 illustrates an example of a process flow 600 that supports breathing disturbance detection and reporting in accordance with aspects of the present disclosure. In some examples, the process flow 600 may implement aspects of the system 100, the system 200, the wearable device diagram 300, the GUI 400, the process flow 500, or any combination thereof. The user device 106-f and the wearable device 104-f may be examples of a user device 106 and a wearable device 104, respectively, as described with reference to FIGS. 1 and 2. Alternative examples of the following may be implemented, where some processes are performed in a different order than described or are not performed. In some cases, processes may include additional features not mentioned below, or further processes may be added.

In some examples, at 605, a user device 106-f may preprocess a training physiological dataset for training one or more neural networks (e.g., or other machine learning models). For example, the user device may filter, normalize, resample, and/or pad the training physiological dataset. The training physiological dataset may include features related to breathing disturbance for one or more users (e.g., which may include a user of a wearable device 106-f). For example, the features may include some or all of a decrease in SpO2 relative to a baseline SpO2 level for the one or more users, a decrease in a pulse wave amplitude relative to a baseline pulse wave amplitude of the one or more users, an increase in movement intensity relative to a baseline movement intensity of the one or more users, a decrease in temperature relative to a baseline temperature of the one or more users, an increase in ambient sound level relative to a baseline ambient sound level, a decrease and subsequent increase in heart rate of the one or more users relative to a baseline heart rate, and so on.

In some examples, at 610, the user device 106-f may train the one or more neural networks using the preprocessed training physiological dataset. For example, the user device 106-f may train the one or more neural networks to identify breathing disturbance (e.g., apnea, hypopnea, oxygen desaturation, RERA) based on identifying some or all of the features. By training the one or more neural networks, the user device 106-f may determine one or more parameters or weights of the one or more neural networks. For example, the neural network may iteratively learn to decrease (e.g., minimize) an error between one or more predicted labels and one or more ground-truth labels associated with the training physiological dataset. In some examples, the ground-truth labels may refer to a human-expert scoring of breathing disturbance events (e.g., apnea, hypopnea, oxygen desaturation, RERA) detected through a sleep period of the one or more users via polysomnography.

In some examples, at 615, the user device 106-f may determine a condition to trigger one or more physiological measurements (e.g., SpO2 measurements, other PPG measurements, temperature measurements, motion measurements, audio measurements) at the wearable device 104-f. For example, the user device 106-f may determine a sleep state of the user, which may trigger the one or more oxygen saturation measurements. The user device 106-e may determine the sleep state of the user based on one or more relationships between the sensor data from the wearable device, the application data, the physiological data from the wearable device, or any combination thereof. For example, at 520, the user device 106-f may select the condition to trigger the SpO2 measurement for the user based on implementing a learning model (e.g., a neural network), as described herein. The user device 106-f may indicate the trigger condition to the wearable ring device 104-f.

At 620, the wearable ring device 104-f may acquire physiological data from the user (e.g., based on detecting that the trigger condition is satisfied). The physiological data may include multiple time series, such as SpO2 data collected via PPG measurements from one or more light-emitting components and one or more light-receiving components of the wearable ring device 104-f, as described herein. Additionally, or alternatively, the time series may include heartbeat data (e.g., IBI data) collected via the PPG measurements or via an electrocardiogram sensor of the wearable ring device 104-f, pulse wave amplitude data collected via the PPG measurements, movement data comprising an intensity of movement of the user collected via one or more accelerometers of the wearable device 104-f, skin temperature data collected via one or more temperature sensors of the wearable ring device 104-f, or audio data collected via a microphone of the wearable ring device 104-f. In some examples, the audio data or one or more other measurements may be collected via another device (e.g., the user device 106-f, a charger of the wearable ring device 104-f).

In some examples, the physiological data (e.g., the multiple time series) may be collected using the same or different sampling frequencies. The time series may have uniform or irregular sampling rates. In some examples, the sampling frequencies/rates may be based on the trigger condition.

At 625, the wearable ring device 104-f may transmit the physiological data (e.g., the time series) to the user device 106-f via one or more electronic signals. In some examples, at 630, the user device 106-f may preprocess the physiological data. For example, the user device may filter, normalize, resample, and/or pad the physiological data. The preprocessing of the physiological data (e.g., and the training physiological dataset) may improve a signal-to-noise ratio (SNR) of data input into the neural networks.

In some examples, at 635, the user device 106-f may select a neural network (e.g., a deep learning neural network). For example, the user device 106-f may select the neural network from the one or more neural networks. Each neural network of the one or more neural networks may include a different set of inputs and may be trained on a corresponding set of features of the training physiological dataset. In some examples, the user device 106-f may select a neural network based on inputs included in the physiological data (e.g., the time series) and/or based on a quality of the physiological data. For example, if one or more of the time series in the physiological data has a quality (e.g., an SNR) that is below a threshold, the user device 106-f may select a neural network that does not include the one or more low-quality time series as inputs.

At 640, the user device 106-f may generate a breathing disturbance metric by inputting some or all of the physiological data (e.g., the time series) into a neural network (e.g., the selected neural network). The neural network may output the breathing disturbance metric (e.g., a probability time series) with a time resolution of one or more seconds, one or more minutes, one or more hours, and so on. In some examples, the neural network may output a single breathing disturbance metric (e.g., representing breathing disturbance for the whole sleep period of the user).

The output probability time series may represent a probability that the user experienced a breathing disturbance event (e.g., apnea, hypopnea, oxygen desaturation, RERA) during one or more time periods corresponding to the time resolution of the probability time series. That is, the breathing disturbance metric may indicate whether the neural network detected a respiratory event (e.g., an apnea event, a hypopnea event, a drop in SpO2 levels) during the one or more time periods (e.g., sleep periods of the user).

In some examples, the user device 106-f may generate information related to the breathing disturbance metric. For example, the user device 106-f may binarize the probability time series (e.g., using a predefined threshold) to determine, based on the probability satisfying the threshold, if a breathing disturbance event occurred. The user device 106-a may compute a metric (e.g., an index) of apnea severity, such as an AHI, ODI, HB, or BDI.

At 645, the user device 106-f may cause a GUI of the user device 106-f to display the information related to the breathing disturbance metric. For example, the GUI may display the generated index (e.g., the AHI, the BDI, the ODI) and/or a timeline of SpO2 or breathing disturbance measurements throughout the sleep periods of the user. In some examples, the user device 106-f may cause the GUI to display a timeline of breathing disturbance, oxygen saturation, or some combination thereof (e.g., based on the binarized probability time series). For example, as described with reference to FIG. 4, the GUI may display a timeline showing times (e.g., timestamps) of the sleep period of the user during which the user may have experienced breathing disturbance events.

In some examples, the process of acquiring physiological data and generating the breathing disturbance metric and/or index may be repeated one or more times (e.g., during one or more sleep periods or nights that the user wears the wearable ring device 104-f). Accordingly, the user may acquire continuous and extended estimates of breathing disturbance severity over time. The user device 106-f may average breathing disturbance metric data collected over multiple sleep periods of the user (e.g., multiple days) to provide a more robust assessment of sleep apnea severity as compared to data collected during a single sleep period. Such longitudinal monitoring of breathing disturbance may allow the user device 106-f to assess an effectiveness of interventions or lifestyle changes (e.g., changes in eating or drinking habits, changes in sleep habits, medication) on the breathing disturbance of the user. The user may therefore use personalized and proactive management strategies to decrease a risk of breathing disturbance events.

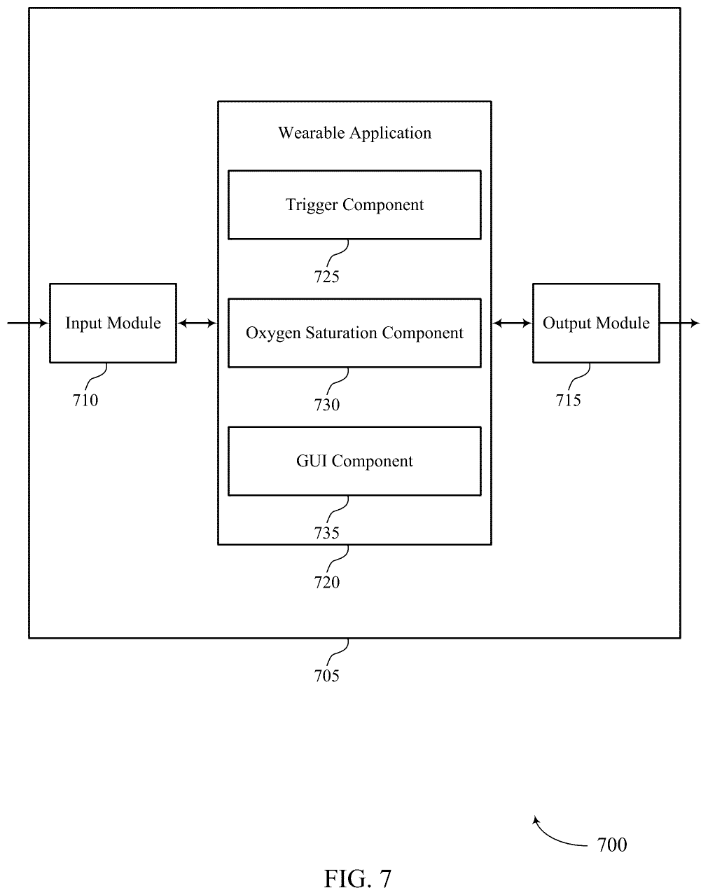

FIG. 7 shows a block diagram 700 of a device 705 that supports oxygen saturation measurement and reporting in accordance with aspects of the present disclosure. The device 705 may include an input module 710, an output module 715, and a wearable application 720. The device 705 may also include a processor. Each of these components may be in communication with one another (e.g., via one or more buses).

The input module 710 may provide a means for receiving information such as packets, user data, control information, or any combination thereof associated with various information channels (e.g., control channels, data channels, information channels related to illness detection techniques). Information may be passed on to other components of the device 705. The input module 710 may utilize a single antenna or a set of multiple antennas.

The output module 715 may provide a means for transmitting signals generated by other components of the device 705. For example, the output module 715 may transmit information such as packets, user data, control information, or any combination thereof associated with various information channels (e.g., control channels, data channels, information channels related to illness detection techniques). In some examples, the output module 715 may be co-located with the input module 710 in a transceiver module. The output module 715 may utilize a single antenna or a set of multiple antennas.

For example, the wearable application 720 may include a trigger component 725, an oxygen saturation component 730, a GUI component 735, or any combination thereof. In some examples, the wearable application 720, or various components thereof, may be configured to perform various operations (e.g., receiving, monitoring, transmitting) using or otherwise in cooperation with the input module 710, the output module 715, or both. For example, the wearable application 720 may receive information from the input module 710, send information to the output module 715, or be integrated in combination with the input module 710, the output module 715, or both to receive information, transmit information, or perform various other operations as described herein.

The wearable application 720 may support performing oxygen saturation measurements from a wearable device in accordance with examples as disclosed herein. The trigger component 725 may be configured as or otherwise support a means for determining a condition to trigger an oxygen saturation measurement for a user associated with the wearable device, wherein the condition corresponds to a physical state of the wearable device, a physiological state of the user, or both, and wherein the condition is determined based at least in part on one or more relationships between sensor data from the wearable device, application data, physiological data from the wearable device, or any combination thereof. The oxygen saturation component 730 may be configured as or otherwise support a means for receiving a measure of oxygen saturation associated with the user from the wearable device based at least in part on the condition. The GUI component 735 may be configured as or otherwise support a means for causing a GUI of a user device to display an indication of the measure of the oxygen saturation for the user.

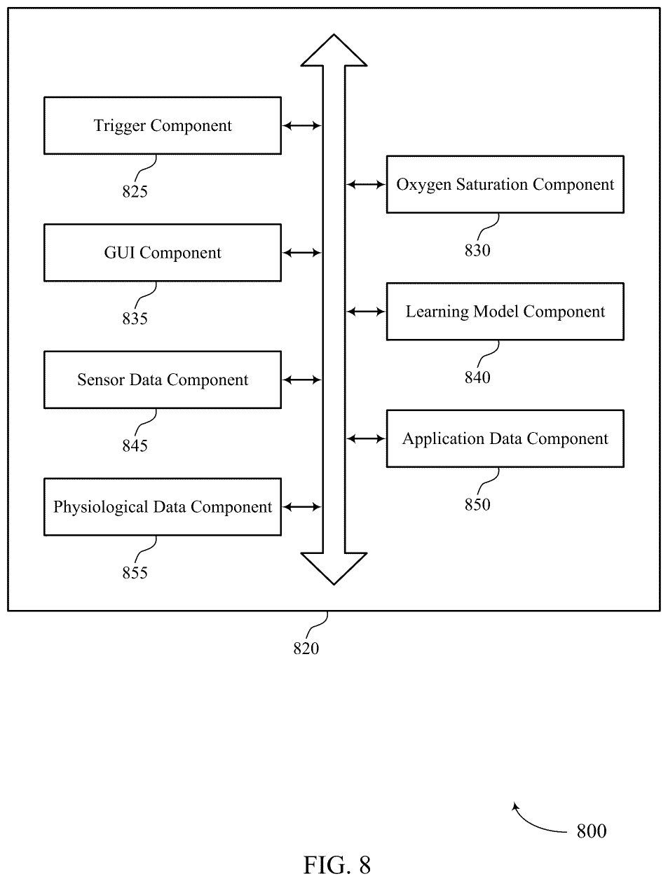

FIG. 8 shows a block diagram 800 of a wearable application 820 that supports oxygen saturation measurement and reporting in accordance with aspects of the present disclosure. The wearable application 820 may be an example of aspects of a wearable application or a wearable application 820, or both, as described herein. The wearable application 820, or various components thereof, may be an example of means for performing various aspects of oxygen saturation measurement and reporting as described herein. For example, the wearable application 820 may include a trigger component 825, an oxygen saturation component 830, a GUI component 835, a learning model component 840, a sensor data component 845, an application data component 850, a physiological data component 855, or any combination thereof. Each of these components may communicate, directly or indirectly, with one another (e.g., via one or more buses).

The wearable application 820 may support performing oxygen saturation measurements from a wearable device in accordance with examples as disclosed herein. The trigger component 825 may be configured as or otherwise support a means for determining a condition to trigger an oxygen saturation measurement for a user associated with the wearable device, wherein the condition corresponds to a physical state of the wearable device, a physiological state of the user, or both, and wherein the condition is determined based at least in part on one or more relationships between sensor data from the wearable device, application data, physiological data from the wearable device, or any combination thereof. The oxygen saturation component 830 may be configured as or otherwise support a means for receiving a measure of oxygen saturation associated with the user from the wearable device based at least in part on the condition. The GUI component 835 may be configured as or otherwise support a means for causing a GUI of a user device to display an indication of the measure of the oxygen saturation for the user.

In some examples, the learning model component 840 may be configured as or otherwise support a means for selecting the condition to trigger the oxygen saturation measurement for the user associated with the wearable device based at least in part on a learning model. In some examples, the learning model includes a mapping between a set of previous oxygen saturation measurements associated with the user, a respective oxygen saturation measurement accuracy associated with each of one or more previous oxygen saturation measurements of the set of previous oxygen saturation measurements, and a set of conditions associated with the set of previous oxygen saturation measurements.

In some examples, the oxygen saturation component 830 may be configured as or otherwise support a means for transmitting a control signal to the wearable device to perform the oxygen saturation measurement for the user associated with the wearable device in accordance with an oxygen saturation measurement cycle based at least in part on the condition. In some examples, receiving the measure of the oxygen saturation associated with the user from the wearable device is based at least in part on the transmitted control signal to the wearable device to perform the oxygen saturation measurement.

In some examples, the learning model component 840 may be configured as or otherwise support a means for selecting the oxygen saturation measurement cycle from a set of oxygen saturation measurement cycles based at least in part on a learning model. In some examples, the learning model includes a mapping between one or more conditions of a set of conditions and one or more oxygen saturation measurement cycles of the set of oxygen saturation measurement cycles. In some examples, transmitting the control signal to the wearable device to perform the oxygen saturation measurement is based at least in part on the selected oxygen saturation measurement cycle. In some examples, the oxygen saturation component 830 may be configured as or otherwise support a means for adjusting the oxygen saturation measurement cycle from a default oxygen saturation measurement cycle based at least in part on the condition. In some examples, the oxygen saturation measurement cycle may include a numerical quantity of oxygen saturation measurements for the wearable device to perform and a periodicity for the wearable device to transmit the measure of the oxygen saturation.

In some examples, the sensor data component 845 may be configured as or otherwise support a means for receiving the sensor data from the wearable device associated with the user. In some examples, the sensor data indicates a position value associated with the wearable device relative to an anatomical feature associated with the user. In some examples, the anatomical feature includes a finger of the user. In some examples, the position value may be indicative of a physical locality of the wearable device on the anatomical feature associated with the user and corresponding to the physical state of the wearable device. In some examples, the sensor data component 845 may be configured as or otherwise support a means for determining that the position value associated with the wearable device relative to the anatomical feature associated with the user satisfies a threshold position value for a threshold duration.

In some examples, the sensor data component 845 may be configured as or otherwise support a means for receiving the sensor data from the wearable device associated with the user. In some examples, the sensor data indicates an orientation value associated with the wearable device relative to an anatomical feature associated with the user. In some examples, the anatomical feature may include a finger of the user. In some examples, the orientation value corresponds to the physical state of the wearable device. In some examples, the sensor data component 845 may be configured as or otherwise support a means for determining that the orientation value associated with the wearable device relative to the anatomical feature associated with the user satisfies a threshold orientation value for a threshold duration.

In some examples, the sensor data component 845 may be configured as or otherwise support a means for receiving the sensor data from the wearable device associated with the user. In some examples, the sensor data indicates a pressure value between the wearable device and an anatomical feature associated with the user. In some examples, the anatomical feature includes a finger of the user. In some examples, the pressure value may be indicative of a force of the wearable device on the anatomical feature associated with the user and corresponding to the physical state of the wearable device. In some examples, the sensor data component 845 may be configured as or otherwise support a means for determining that the pressure value between the wearable device and the anatomical feature associated with the user satisfies a threshold pressure value for a threshold duration.

In some examples, the sensor data component 845 may be configured as or otherwise support a means for receiving the sensor data from the user device associated with the user. In some examples, the sensor data indicates at least one sound characteristic associated with the user. In some examples, the sensor data component 845 may be configured as or otherwise support a means for determining that the at least one sound characteristic associated with the user satisfies a threshold value.

In some examples, the application data component 850 may be configured as or otherwise support a means for receiving the application data, from the user device associated with the user, via one or more applications executable on the user device. In some examples, the one or more applications executable on the user device includes a lifestyle application, a social media application, a utility application, an information outlet application, or any combination thereof. In some examples, the application data is indicative of an activity the user is engaged in or a location of the user, or any combination thereof. In some examples, the application data component 850 may be configured as or otherwise support a means for determining the activity the user is engaged in or the location of the user, or any combination thereof.

In some examples, the physiological data component 855 may be configured as or otherwise support a means for receiving the physiological data associated with the user from the wearable device. In some examples, the physiological data component 855 may be configured as or otherwise support a means for determining that one or more physiological parameters of the received physiological data satisfies a threshold.

In some examples, at least one physiological parameter of the physiological data includes heart rate data associated with the user, the heart rate data corresponding to the physiological state of the user, and the physiological data component 855 may be configured as or otherwise support a means for detecting an abnormal heart rate associated with the user based at least in part on the heart rate data. In some examples, the abnormal heart rate corresponds to a heart rate associated with the user different from a range of heart rate values associated with the user. In some examples, the means for determining the condition may include means for detecting the abnormal heart rate associated with the user.

In some examples, the physiological data component 855 may be configured as or otherwise support a means for collecting the physiological data associated with the user from the wearable device based at least in part on a sleep state of the user. In some examples, the physiological data component 855 may be configured as or otherwise support a means for detecting the sleep state of the user.

In some examples, the physiological data component 855 may be configured as or otherwise support a means for determining one or more breathing disturbances associated with the user during a duration associated with the sleep state of the user based at least in part on triggering the oxygen saturation measurement for the user during the duration associated with the sleep state of the user. In some examples, the GUI component 835 may be configured as or otherwise support a means for causing the GUI of the user device to display the one or more breathing disturbances for the user.

In some examples, to support causing the GUI to display the indication of the measure of the oxygen saturation, the GUI component 835 may be configured as or otherwise support a means for causing the GUI of the user device to display an average breathing regularity during the duration associated with the sleep state of the user, a description of the average breathing regularity, a respective timestamp associated with each of the one or more breathing disturbances, or a magnitude associated with each of the one or more breathing disturbances, or any combination thereof.

In some examples, the trigger component 825 may be configured as or otherwise support a means for receiving an input to trigger the oxygen saturation measurement for the user associated with the wearable device based at least in part on a user input or a setting executable via an application running on the user device. In some examples, the wearable device includes a wearable ring device.

In some examples, the oxygen saturation component 830 may be configured as or otherwise support a means for determining a first timestamp of a previously performed oxygen saturation measurement operation associated with the user device, the wearable device, or both. In some examples, the oxygen saturation component 830 may be configured as or otherwise support a means for determining that a duration between the first timestamp of the previously performed oxygen saturation measurement operation and a second timestamp associated with the determining of the condition to enable the oxygen saturation measurement satisfies a threshold.

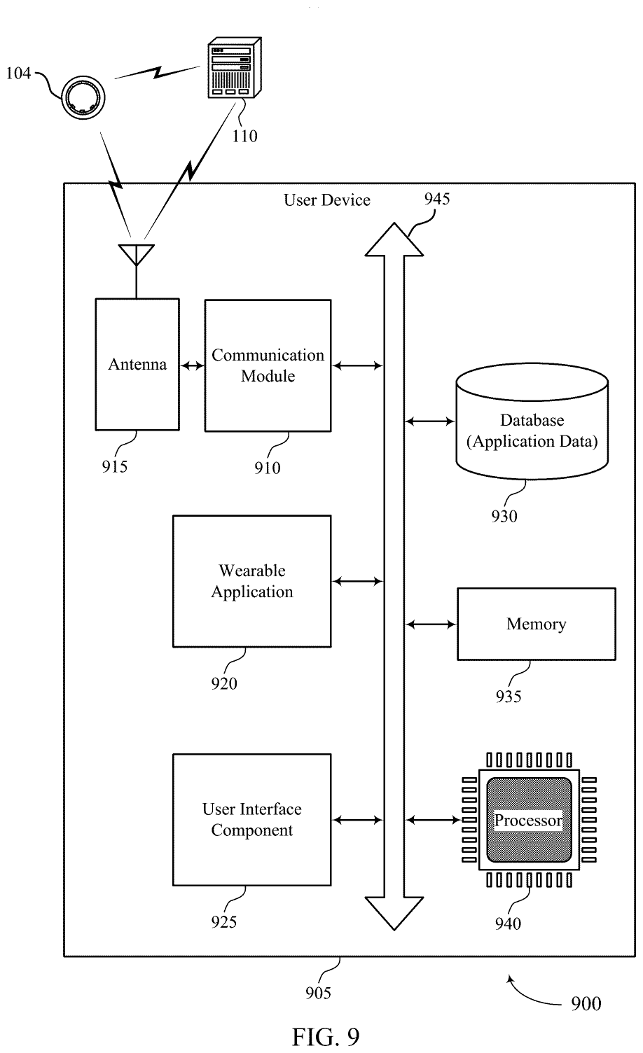

FIG. 9 shows a diagram of a system 900 including a device 905 that supports oxygen saturation measurement and reporting in accordance with aspects of the present disclosure. The device 905 may be an example of or include the components of a device 705 as described herein. The device 905 may include an example of a user device 106, as described previously herein. The device 905 may include components for bi-directional communications including components for transmitting and receiving communications with a wearable device 104 and a server 110, such as a wearable application 920, a communication module 910, an antenna 915, a user interface component 925, a database (application data) 930, a memory 935, and a processor 940. These components may be in electronic communication or otherwise coupled (e.g., operatively, communicatively, functionally, electronically, electrically) via one or more buses (e.g., a bus 945).

The communication module 910 may manage input and output signals for the device 905 via the antenna 915. The communication module 910 may include an example of the communication module 220-b of the user device 106 shown and described in FIG. 2. In this regard, the communication module 910 may manage communications with the ring 104 and the server 110, as illustrated in FIG. 2. The communication module 910 may also manage peripherals not integrated into the device 905. In some cases, the communication module 910 may represent a physical connection or port to an external peripheral. In some cases, the communication module 910 may utilize an operating system such as iOS®, ANDROID®, MS-DOS®, MS-WINDOWS®, OS/2®, UNIX®, LINUX®, or another known operating system. In other cases, the communication module 910 may represent or interact with a wearable device (e.g., ring 104), modem, a keyboard, a mouse, a touchscreen, or a similar device. In some cases, the communication module 910 may be implemented as part of the processor 940. In some examples, a user may interact with the device 905 via the communication module 910, user interface component 925, or via hardware components controlled by the communication module 910.

In some cases, the device 905 may include a single antenna 915. However, in some other cases, the device 905 may have more than one antenna 915, which may be capable of concurrently transmitting or receiving multiple wireless transmissions. The communication module 910 may communicate bi-directionally, via the one or more antennas 915, wired, or wireless links as described herein. For example, the communication module 910 may represent a wireless transceiver and may communicate bi-directionally with another wireless transceiver. The communication module 910 may also include a modem to modulate the packets, to provide the modulated packets to one or more antennas 915 for transmission, and to demodulate packets received from the one or more antennas 915.

The user interface component 925 may manage data storage and processing in a database 930. In some cases, a user may interact with the user interface component 925. In other cases, the user interface component 925 may operate automatically without user interaction. The database 930 may be an example of a single database, a distributed database, multiple distributed databases, a data store, a data lake, or an emergency backup database.

The memory 935 may include RAM and ROM. The memory 935 may store computer-readable, computer-executable software including instructions that, when executed, cause the processor 940 to perform various functions described herein. In some cases, the memory 935 may contain, among other things, a BIOS which may control basic hardware or software operation such as the interaction with peripheral components or devices.

The processor 940 may include an intelligent hardware device, (e.g., a general-purpose processor, a DSP, a CPU, a microcontroller, an ASIC, an FPGA, a programmable logic device, a discrete gate or transistor logic component, a discrete hardware component, or any combination thereof). In some cases, the processor 940 may be configured to operate a memory array using a memory controller. In other cases, a memory controller may be integrated into the processor 940. The processor 940 may be configured to execute computer-readable instructions stored in a memory 935 to perform various functions (e.g., functions or tasks supporting a method and system for sleep staging algorithms).

The wearable application 920 may support performing oxygen saturation measurements from a wearable device in accordance with examples as disclosed herein. For example, the wearable application 920 may be configured as or otherwise support a means for determining a condition to trigger an oxygen saturation measurement for a user associated with the wearable device, wherein the condition corresponds to a physical state of the wearable device, a physiological state of the user, or both, and wherein the condition is determined based at least in part on one or more relationships between sensor data from the wearable device, application data, physiological data from the wearable device, or any combination thereof. The wearable application 920 may be configured as or otherwise support a means for receiving a measure of oxygen saturation associated with the user from the wearable device based at least in part on the condition. The wearable application 920 may be configured as or otherwise support a means for causing a GUI of the device 905 (e.g., a user device) to display an indication of the measure of the oxygen saturation for the user.

By including or configuring the wearable application 920 in accordance with examples as described herein, the device 905 may support techniques for the device 905 (e.g., a user device) to trigger one or more measures of oxygen saturation at a wearable device, which may reduce power consumption at the wearable device, improve accuracy of measurements at the wearable device, or the like.

The wearable application 920 may include an application (e.g., “app”), program, software, or other component which is configured to facilitate communications with a ring 104, server 110, other user devices 106, and the like. For example, the wearable application 920 may include an application executable on a user device 106 which is configured to receive data (e.g., physiological data) from a ring 104, perform processing operations on the received data, transmit and receive data with the servers 110, and cause presentation of data to a user 102.

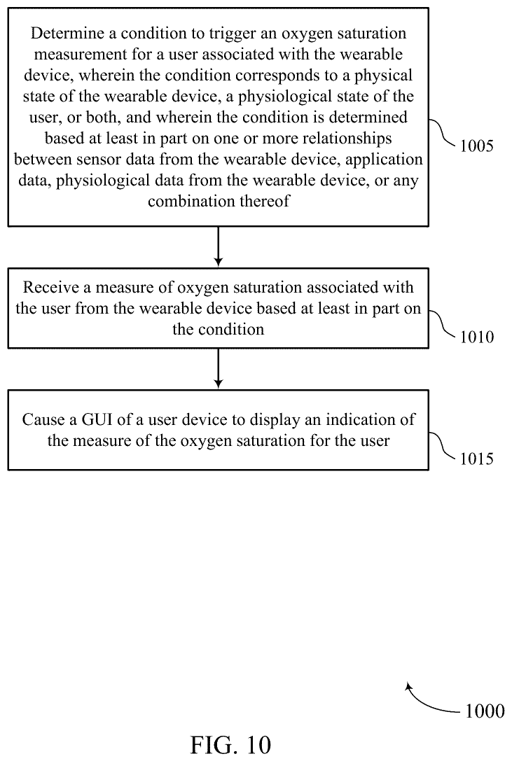

FIG. 10 shows a flowchart illustrating a method 1000 that supports oxygen saturation measurement and reporting in accordance with aspects of the present disclosure. The operations of the method 1000 may be implemented by a user device or its components as described herein. For example, the operations of the method 1000 may be performed by a user device as described with reference to FIGS. 1 through 9. In some examples, a user device may execute a set of instructions to control the functional elements of the user device to perform the described functions. Additionally, or alternatively, a user device may perform aspects of the described functions using special-purpose hardware.

At 1005, the method may include determining a condition to trigger an oxygen saturation measurement for a user associated with the wearable device, wherein the condition corresponds to a physical state of the wearable device, a physiological state of the user, or both, and wherein the condition is determined based at least in part on one or more relationships between sensor data from the wearable device, application data, physiological data from the wearable device, or any combination thereof. The operations of 1005 may be performed in accordance with examples as disclosed herein. In some examples, aspects of the operations of 1005 may be performed by a trigger component 725 as described with reference to FIG. 7.

At 1010, the method may include receiving a measure of oxygen saturation associated with the user from the wearable device based at least in part on the condition. The operations of 1010 may be performed in accordance with examples as disclosed herein. In some examples, aspects of the operations of 1010 may be performed by an oxygen saturation component 730 as described with reference to FIG. 7.

At 1015, the method may include causing a GUI of a user device to display an indication of the measure of the oxygen saturation for the user. The operations of 1015 may be performed in accordance with examples as disclosed herein. In some examples, aspects of the operations of 1015 may be performed by a GUI component 735 as described with reference to FIG. 7.

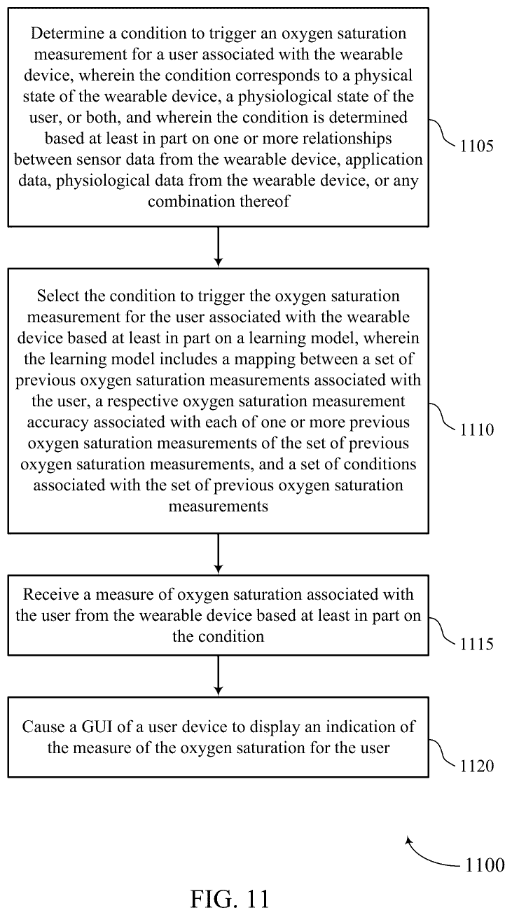

FIG. 11 shows a flowchart illustrating a method 1100 that supports oxygen saturation measurement and reporting in accordance with aspects of the present disclosure. The operations of the method 1100 may be implemented by a user device or its components as described herein. For example, the operations of the method 1100 may be performed by a user device as described with reference to FIGS. 1 through 9. In some examples, a user device may execute a set of instructions to control the functional elements of the user device to perform the described functions. Additionally, or alternatively, a user device may perform aspects of the described functions using special-purpose hardware.

At 1105, the method may include determining a condition to trigger an oxygen saturation measurement for a user associated with the wearable device, wherein the condition corresponds to a physical state of the wearable device, a physiological state of the user, or both, and wherein the condition is determined based at least in part on one or more relationships between sensor data from the wearable device, application data, physiological data from the wearable device, or any combination thereof. The operations of 1105 may be performed in accordance with examples as disclosed herein. In some examples, aspects of the operations of 1105 may be performed by a trigger component 825 as described with reference to FIG. 8.

At 1110, the method may include selecting the condition to trigger the oxygen saturation measurement for the user associated with the wearable device based at least in part on a learning model, wherein the learning model includes a mapping between a set of previous oxygen saturation measurements associated with the user, a respective oxygen saturation measurement accuracy associated with each of one or more previous oxygen saturation measurements of the set of previous oxygen saturation measurements, and a set of conditions associated with the set of previous oxygen saturation measurements. The operations of 1110 may be performed in accordance with examples as disclosed herein. In some examples, aspects of the operations of 1110 may be performed by a learning model component 840 as described with reference to FIG. 8.

At 1115, the method may include receiving a measure of oxygen saturation associated with the user from the wearable device based at least in part on the condition. The operations of 1115 may be performed in accordance with examples as disclosed herein. In some examples, aspects of the operations of 1115 may be performed by an oxygen saturation component 830 as described with reference to FIG. 8.

At 1120, the method may include causing a GUI of a user device to display an indication of the measure of the oxygen saturation for the user. The operations of 1120 may be performed in accordance with examples as disclosed herein. In some examples, aspects of the operations of 1120 may be performed by a GUI component 835 as described with reference to FIG. 8.

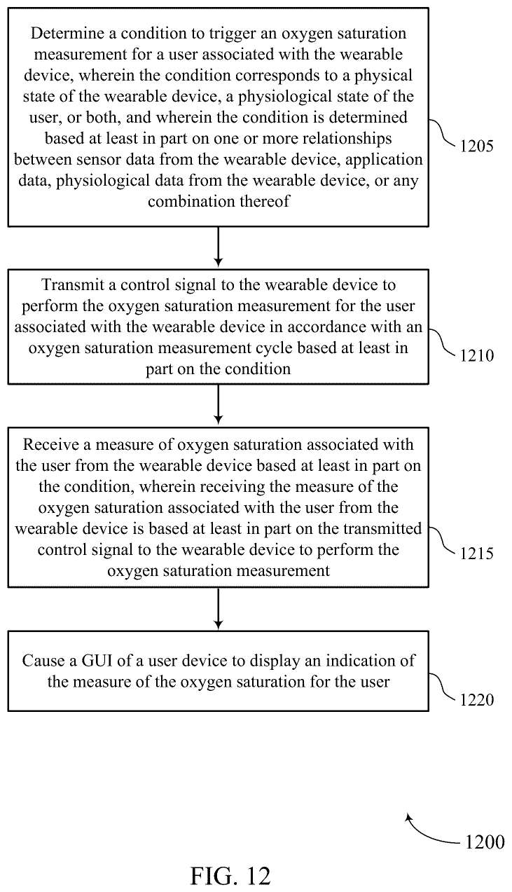

FIG. 12 shows a flowchart illustrating a method 1200 that supports oxygen saturation measurement and reporting in accordance with aspects of the present disclosure. The operations of the method 1200 may be implemented by a user device or its components as described herein. For example, the operations of the method 1200 may be performed by a user device as described with reference to FIGS. 1 through 9. In some examples, a user device may execute a set of instructions to control the functional elements of the user device to perform the described functions. Additionally, or alternatively, a user device may perform aspects of the described functions using special-purpose hardware.

At 1205, the method may include determining a condition to trigger an oxygen saturation measurement for a user associated with the wearable device, wherein the condition corresponds to a physical state of the wearable device, a physiological state of the user, or both, and wherein the condition is determined based at least in part on one or more relationships between sensor data from the wearable device, application data, physiological data from the wearable device, or any combination thereof. The operations of 1205 may be performed in accordance with examples as disclosed herein. In some examples, aspects of the operations of 1205 may be performed by a trigger component 725 as described with reference to FIG. 7.

At 1210, the method may include transmitting a control signal to the wearable device to perform the oxygen saturation measurement for the user associated with the wearable device in accordance with an oxygen saturation measurement cycle based at least in part on the condition. The operations of 1210 may be performed in accordance with examples as disclosed herein. In some examples, aspects of the operations of 1210 may be performed by an oxygen saturation component 730 as described with reference to FIG. 7.

At 1215, the method may include receiving a measure of oxygen saturation associated with the user from the wearable device based at least in part on the condition, wherein receiving the measure of the oxygen saturation associated with the user from the wearable device is based at least in part on the transmitted control signal to the wearable device to perform the oxygen saturation measurement. The operations of 1215 may be performed in accordance with examples as disclosed herein. In some examples, aspects of the operations of 1215 may be performed by an oxygen saturation component 730 as described with reference to FIG. 7.

At 1220, the method may include causing a GUI of a user device to display an indication of the measure of the oxygen saturation for the user. The operations of 1220 may be performed in accordance with examples as disclosed herein. In some examples, aspects of the operations of 1220 may be performed by a GUI component 735 as described with reference to FIG. 7.

It should be noted that the methods described above describe possible implementations, and that the operations and the steps may be rearranged or otherwise modified and that other implementations are possible. Furthermore, aspects from two or more of the methods may be combined.

A method by an apparatus is described. The method may include a wearable ring device configured to acquire physiological data from a user, the physiological data comprising oxygen saturation data collected via photoplethysmogram (PPG) measurements from one or more light-emitting components and one or more light-receiving components of the wearable ring device, heartbeat data collected via the PPG measurements from the one or more light-emitting components and the one or more light-receiving components of the wearable ring device, heartbeat data collected via an electrocardiogram sensor of the wearable ring device, pulse wave amplitude data collected via the PPG measurements, movement data comprising an intensity of movement of the user collected via one or more accelerometers of the wearable ring device, temperature data collected via one or more temperature sensors of the wearable ring device, or some combination thereof, wherein the physiological data is collected throughout a time interval that includes one or more sleep periods of the user, one or more processors communicatively coupled with the wearable ring device, wherein the one or more processors are configured to, receive the physiological data acquired via the wearable ring device via one or more electronic signals, input the physiological data into a neural network, the neural network trained to detect breathing disturbance events based on a plurality of features within a training physiological dataset associated with a plurality of users, the plurality of features comprising a first decrease in an oxygen saturation level relative to a baseline oxygen saturation level of the plurality of users, a second decrease in a pulse wave amplitude relative to a baseline heartbeat amplitude of the plurality of users, an increase in intensity of movement relative to a baseline intensity of the plurality of users, a third decrease in temperature data relative to baseline temperature data of the plurality of users, or some combination thereof, generate, using the neural network, a breathing disturbance metric based at least in part on the plurality of features within the physiological data during the time interval, the breathing disturbance metric associated with a probability that the user experienced a breathing disturbance event during the time interval, and transmit an instruction to a graphical user interface (GUI) of a user device associated with the wearable ring device, the instruction configured to cause the GUI to display information associated with the breathing disturbance metric.

An apparatus is described. The apparatus may include one or more memories storing processor executable code, and one or more processors coupled with the one or more memories. The one or more processors may individually or collectively be operable to execute the code to cause the apparatus to a wearable ring device configure to acquire physiological data from a user, the physiological data comprising oxygen saturation data collected via photoplethysmogram (PPG) measurements from one or more light-emitting components and one or more light-receiving components of the wearable ring device, heartbeat data collected via the PPG measurements from the one or more light-emitting components and the one or more light-receiving components of the wearable ring device, heartbeat data collected via an electrocardiogram sensor of the wearable ring device, pulse wave amplitude data collected via the PPG measurements, movement data comprising an intensity of movement of the user collected via one or more accelerometers of the wearable ring device, temperature data collected via one or more temperature sensors of the wearable ring device, or some combination thereof, wherein the physiological data is collected throughout a time interval that includes one or more sleep periods of the user, one or more processors communicatively couple with the wearable ring device, wherein the one or more processors are configured to, receive the physiological data acquired via the wearable ring device via one or more electronic signals, input the physiological data into a neural network, the neural network trained to detect breathing disturbance events based on a plurality of features within a training physiological dataset associated with a plurality of users, the plurality of features comprising a first decrease in an oxygen saturation level relative to a baseline oxygen saturation level of the plurality of users, a second decrease in a pulse wave amplitude relative to a baseline heartbeat amplitude of the plurality of users, an increase in intensity of movement relative to a baseline intensity of the plurality of users, a third decrease in temperature data relative to baseline temperature data of the plurality of users, or some combination thereof, generate, using the neural network, a breathing disturbance metric based at least in part on the plurality of features within the physiological data during the time interval, the breathing disturbance metric associated with a probability that the user experienced a breathing disturbance event during the time interval, and transmit an instruction to a graphical user interface (GUI) of a user device associated with the wearable ring device, the instruction configured to cause the GUI to display information associated with the breathing disturbance metric.

Another apparatus is described. The apparatus may include means for a wearable ring device configured to acquire physiological data from a user, the physiological data comprising oxygen saturation data collected via photoplethysmogram (PPG) measurements from one or more light-emitting components and one or more light-receiving components of the wearable ring device, heartbeat data collected via the PPG measurements from the one or more light-emitting components and the one or more light-receiving components of the wearable ring device, heartbeat data collected via an electrocardiogram sensor of the wearable ring device, pulse wave amplitude data collected via the PPG measurements, movement data comprising an intensity of movement of the user collected via one or more accelerometers of the wearable ring device, temperature data collected via one or more temperature sensors of the wearable ring device, or some combination thereof, wherein the physiological data is collected throughout a time interval that includes one or more sleep periods of the user, means for one or more processors communicatively coupled with the wearable ring device, wherein the one or more processors are configured to, means for receive the physiological data acquired via the wearable ring device via one or more electronic signals, means for input the physiological data into a neural network, the neural network trained to detect breathing disturbance events based on a plurality of features within a training physiological dataset associated with a plurality of users, the plurality of features comprising a first decrease in an oxygen saturation level relative to a baseline oxygen saturation level of the plurality of users, a second decrease in a pulse wave amplitude relative to a baseline heartbeat amplitude of the plurality of users, an increase in intensity of movement relative to a baseline intensity of the plurality of users, a third decrease in temperature data relative to baseline temperature data of the plurality of users, or some combination thereof, means for generate, using the neural network, a breathing disturbance metric based at least in part on the plurality of features within the physiological data during the time interval, the breathing disturbance metric associated with a probability that the user experienced a breathing disturbance event during the time interval, and means for transmit an instruction to a graphical user interface (GUI) of a user device associated with the wearable ring device, the instruction configured to cause the GUI to display information associated with the breathing disturbance metric.

A non-transitory computer-readable medium storing code is described. The code may include instructions executable by one or more processors to a wearable ring device configure to acquire physiological data from a user, the physiological data comprising oxygen saturation data collected via photoplethysmogram (PPG) measurements from one or more light-emitting components and one or more light-receiving components of the wearable ring device, heartbeat data collected via the PPG measurements from the one or more light-emitting components and the one or more light-receiving components of the wearable ring device, heartbeat data collected via an electrocardiogram sensor of the wearable ring device, pulse wave amplitude data collected via the PPG measurements, movement data comprising an intensity of movement of the user collected via one or more accelerometers of the wearable ring device, temperature data collected via one or more temperature sensors of the wearable ring device, or some combination thereof, wherein the physiological data is collected throughout a time interval that includes one or more sleep periods of the user, one or more processors communicatively couple with the wearable ring device, wherein the one or more processors are configured to, receive the physiological data acquired via the wearable ring device via one or more electronic signals, input the physiological data into a neural network, the neural network trained to detect breathing disturbance events based on a plurality of features within a training physiological dataset associated with a plurality of users, the plurality of features comprising a first decrease in an oxygen saturation level relative to a baseline oxygen saturation level of the plurality of users, a second decrease in a pulse wave amplitude relative to a baseline heartbeat amplitude of the plurality of users, an increase in intensity of movement relative to a baseline intensity of the plurality of users, a third decrease in temperature data relative to baseline temperature data of the plurality of users, or some combination thereof, generate, using the neural network, a breathing disturbance metric based at least in part on the plurality of features within the physiological data during the time interval, the breathing disturbance metric associated with a probability that the user experienced a breathing disturbance event during the time interval, and transmit an instruction to a graphical user interface (GUI) of a user device associated with the wearable ring device, the instruction configured to cause the GUI to display information associated with the breathing disturbance metric.

Some examples of the method, apparatus, and non-transitory computer-readable medium described herein may further include operations, features, means, or instructions for determine a condition to trigger acquisition of the physiological data from the user associated with the wearable ring device, wherein the condition corresponds to a physical state of the wearable ring device, a physiological state of the user, or both, and wherein the condition may be determined based at least in part on one or more relationships between sensor data from the wearable ring device, application data, physiological data from the wearable ring device, or any combination thereof, receive the oxygen saturation data associated with the user from the wearable ring device based at least in part on the condition, and cause the GUI to display an indication of the oxygen saturation data for the user.

Some examples of the method, apparatus, and non-transitory computer-readable medium described herein may further include operations, features, means, or instructions for acquiring the physiological data based at least in part on a sleep state of the user, wherein determining the condition comprises and detecting the sleep state of the user.

In some examples of the method, apparatus, and non-transitory computer-readable medium described herein, the breathing disturbance metric comprises a probability of an apnea event, a probability of a hypopnea event, a probability of an oxygen desaturation event, a probability of a respiratory effort-related arousal event, or some combination thereof.

In some examples of the method, apparatus, and non-transitory computer-readable medium described herein, the information associated with the breathing disturbance metric comprises a breathing disturbance index, an apnea-hypopnea index, an oxygen desaturation index, or some combination thereof.

Some examples of the method, apparatus, and non-transitory computer-readable medium described herein may further include operations, features, means, or instructions for transmit an instruction to the GUI to cause the GUI to display a timeline associated with the one or more sleep periods of the user, wherein the timeline comprises one or more breathing disturbance events and a timestamp associated with the one or more breathing disturbance events.

Some examples of the method, apparatus, and non-transitory computer-readable medium described herein may further include operations, features, means, or instructions for select the neural network from a set of neural networks, wherein each neural network of the set of neural networks may be associated with one or more combinations of physiological data.

Some examples of the method, apparatus, and non-transitory computer-readable medium described herein may further include operations, features, means, or instructions for perform a preprocessing of the training physiological dataset and of the physiological data, wherein the preprocessing comprises filtering the training physiological dataset and the physiological data, normalizing the training physiological dataset and the physiological data, resampling the training physiological dataset and the physiological data, padding the training physiological dataset and the physiological data, or some combination thereof and train the neural network based at least in part on the preprocessed training physiological dataset.

In some examples of the method, apparatus, and non-transitory computer-readable medium described herein, training the neural network may include operations, features, means, or instructions for determining one or more parameter values associated with the neural network based at least in part on an error between one or more ground-truth labels associated with the preprocessed training physiological dataset and one or more predicted labels output from the neural network.

Some examples of the method, apparatus, and non-transitory computer-readable medium described herein may further include operations, features, means, or instructions for generate the breathing disturbance metric periodically during the time interval, wherein a periodicity of the breathing disturbance metric may be uniform or irregular.

Some examples of the method, apparatus, and non-transitory computer-readable medium described herein may further include operations, features, means, or instructions for input an auditory measurement into the neural network, the auditory measurement comprising a measurement of an ambient sound level during the time interval, wherein the plurality of features within the training physiological dataset further comprise an increase in ambient sound level relative to a baseline ambient sound level.

Some examples of the method, apparatus, and non-transitory computer-readable medium described herein may further include operations, features, means, or instructions for perform the auditory measurement during the time interval via a microphone of the wearable ring device, via a charger of the wearable ring device, or via the user device.

A method for performing oxygen saturation measurements from a wearable device is described. The method may include determining a condition to trigger an oxygen saturation measurement for a user associated with the wearable device, wherein the condition corresponds to a physical state of the wearable device, a physiological state of the user, or both, and wherein the condition is determined based at least in part on one or more relationships between sensor data from the wearable device, application data, physiological data from the wearable device, or any combination thereof, receiving a measure of oxygen saturation associated with the user from the wearable device based at least in part on the condition, and causing a GUI of a user device to display an indication of the measure of the oxygen saturation for the user.

An apparatus for performing oxygen saturation measurements from a wearable device is described. The apparatus may include a processor, memory coupled with the processor, and instructions stored in the memory. The instructions may be executable by the processor to cause the apparatus to determine a condition to trigger an oxygen saturation measurement for a user associated with the wearable device, wherein the condition corresponds to a physical state of the wearable device, a physiological state of the user, or both, and wherein the condition is determined based at least in part on one or more relationships between sensor data from the wearable device, application data, physiological data from the wearable device, or any combination thereof, receive a measure of oxygen saturation associated with the user from the wearable device based at least in part on the condition, and cause a GUI of a user device to display an indication of the measure of the oxygen saturation for the user.

Another apparatus for performing oxygen saturation measurements from a wearable device is described. The apparatus may include means for determining a condition to trigger an oxygen saturation measurement for a user associated with the wearable device, wherein the condition corresponds to a physical state of the wearable device, a physiological state of the user, or both, and wherein the condition is determined based at least in part on one or more relationships between sensor data from the wearable device, application data, physiological data from the wearable device, or any combination thereof, means for receiving a measure of oxygen saturation associated with the user from the wearable device based at least in part on the condition, and means for causing a GUI of a user device to display an indication of the measure of the oxygen saturation for the user.

A non-transitory computer-readable medium storing code for performing oxygen saturation measurements from a wearable device is described. The code may include instructions executable by a processor to determine a condition to trigger an oxygen saturation measurement for a user associated with the wearable device, wherein the condition corresponds to a physical state of the wearable device, a physiological state of the user, or both, and wherein the condition is determined based at least in part on one or more relationships between sensor data from the wearable device, application data, physiological data from the wearable device, or any combination thereof, receive a measure of oxygen saturation associated with the user from the wearable device based at least in part on the condition, and cause a GUI of a user device to display an indication of the measure of the oxygen saturation for the user.

Some examples of the method, apparatuses, and non-transitory computer-readable medium described herein may further include operations, features, means, or instructions for selecting the condition to trigger the oxygen saturation measurement for the user associated with the wearable device based at least in part on a learning model, wherein the learning model comprises a mapping between a set of previous oxygen saturation measurements associated with the user, a respective oxygen saturation measurement accuracy associated with each of one or more previous oxygen saturation measurements of the set of previous oxygen saturation measurements, and a set of conditions associated with the set of previous oxygen saturation measurements.

Some examples of the method, apparatuses, and non-transitory computer-readable medium described herein may further include operations, features, means, or instructions for transmitting a control signal to the wearable device to perform the oxygen saturation measurement for the user associated with the wearable device in accordance with an oxygen saturation measurement cycle based at least in part on the condition, wherein receiving the measure of the oxygen saturation associated with the user from the wearable device may be based at least in part on the transmitted control signal to the wearable device to perform the oxygen saturation measurement.

Some examples of the method, apparatuses, and non-transitory computer-readable medium described herein may further include operations, features, means, or instructions for selecting the oxygen saturation measurement cycle from a set of oxygen saturation measurement cycles based at least in part on a learning model, wherein the learning model comprises a mapping between one or more conditions of a set of conditions and one or more oxygen saturation measurement cycles of the set of oxygen saturation measurement cycles, and wherein transmitting the control signal to the wearable device to perform the oxygen saturation measurement may be based at least in part on the selected oxygen saturation measurement cycle.

Some examples of the method, apparatuses, and non-transitory computer-readable medium described herein may further include operations, features, means, or instructions for adjusting the oxygen saturation measurement cycle from a default oxygen saturation measurement cycle based at least in part on the condition.

In some examples, the oxygen saturation measurement cycle may include a numerical quantity of oxygen saturation measurements for the wearable device to perform and a periodicity for the wearable device to transmit the measure of the oxygen saturation.

Some examples of the method, apparatuses, and non-transitory computer-readable medium described herein may further include operations, features, means, or instructions for receiving the sensor data from the wearable device associated with the user, wherein the sensor data indicates a position value associated with the wearable device relative to an anatomical feature associated with the user, the anatomical feature comprising a finger of the user, the position value being indicative of a physical locality of the wearable device on the anatomical feature associated with the user and corresponding to the physical state of the wearable device, wherein determining the condition comprises determining that the position value associated with the wearable device relative to the anatomical feature associated with the user satisfies a threshold position value for a threshold duration.

Some examples of the method, apparatuses, and non-transitory computer-readable medium described herein may further include operations, features, means, or instructions for receiving the sensor data from the wearable device associated with the user, wherein the sensor data indicates an orientation value associated with the wearable device relative to an anatomical feature associated with the user, the anatomical feature comprising a finger of the user, the orientation value corresponding to the physical state of the wearable device, wherein determining the condition comprises determining that the orientation value associated with the wearable device relative to the anatomical feature associated with the user satisfies a threshold orientation value for a threshold duration.

Some examples of the method, apparatuses, and non-transitory computer-readable medium described herein may further include operations, features, means, or instructions for receiving the sensor data from the wearable device associated with the user, wherein the sensor data indicates a pressure value between the wearable device and an anatomical feature associated with the user, the anatomical feature comprising a finger of the user, the pressure value being indicative of a force of the wearable device on the anatomical feature associated with the user and corresponding to the physical state of the wearable device, wherein determining the condition comprises determining that the pressure value between the wearable device and the anatomical feature associated with the user satisfies a threshold pressure value for a threshold duration.

Some examples of the method, apparatuses, and non-transitory computer-readable medium described herein may further include operations, features, means, or instructions for receiving the sensor data from the user device associated with the user, wherein the sensor data indicates at least one sound characteristic associated with the user, wherein determining the condition comprises determining that the at least one sound characteristic associated with the user satisfies a threshold value.

Some examples of the method, apparatuses, and non-transitory computer-readable medium described herein may further include operations, features, means, or instructions for receiving the application data, from the user device associated with the user, via one or more applications executable on the user device, the one or more applications executable on the user device comprising a lifestyle application, a social media application, a utility application, an information outlet application, or any combination thereof, and wherein the application data may be indicative of an activity the user may be engaged in or a location of the user, or any combination thereof, wherein determining the condition comprises determining the activity the user may be engaged in or the location of the user, or any combination thereof.

Some examples of the method, apparatuses, and non-transitory computer-readable medium described herein may further include operations, features, means, or instructions for receiving the physiological data associated with the user from the wearable device, wherein determining the condition comprises determining that one or more physiological parameters of the received physiological data satisfies a threshold.

In some examples of the method, apparatuses, and non-transitory computer-readable medium described herein, where at least one physiological parameter of the physiological data comprises heart rate data associated with the user, the heart rate data corresponding to the physiological state of the user and the method, apparatuses, and non-transitory computer-readable medium may include further operations, features, means, or instructions for detecting an abnormal heart rate associated with the user based at least in part on the heart rate data, wherein the abnormal heart rate corresponds to a heart rate associated with the user different from a range of heart rate values associated with the user, wherein determining the condition comprises detecting the abnormal heart rate associated with the user.

Some examples of the method, apparatuses, and non-transitory computer-readable medium described herein may further include operations, features, means, or instructions for collecting the physiological data associated with the user from the wearable device based at least in part on a sleep state of the user, wherein determining the condition comprises detecting the sleep state of the user.

Some examples of the method, apparatuses, and non-transitory computer-readable medium described herein may further include operations, features, means, or instructions for determining one or more breathing disturbances associated with the user during a duration associated with the sleep state of the user based at least in part on triggering the oxygen saturation measurement for the user during the duration associated with the sleep state of the user, wherein causing the GUI to display the indication of the measure of the oxygen saturation comprises causing the GUI of the user device to display the one or more breathing disturbances for the user.

In some examples of the method, apparatuses, and non-transitory computer-readable medium described herein, causing the GUI to display the indication of the measure of the oxygen saturation may include operations, features, means, or instructions for causing the GUI of the user device to display an average breathing regularity during the duration associated with the sleep state of the user, a description of the average breathing regularity, a respective timestamp associated with each of the one or more breathing disturbances, or a magnitude associated with each of the one or more breathing disturbances, or any combination thereof.

Some examples of the method, apparatuses, and non-transitory computer-readable medium described herein may further include operations, features, means, or instructions for receiving an input to trigger the oxygen saturation measurement for the user associated with the wearable device based at least in part on a user input or a setting executable via an application running on the user device.

In some examples of the method, apparatuses, and non-transitory computer-readable medium described herein, the wearable device comprises a wearable ring device.

Some examples of the method, apparatuses, and non-transitory computer-readable medium described herein may further include operations, features, means, or instructions for determining a first timestamp of a previously performed oxygen saturation measurement operation associated with the user device, the wearable device, or both, wherein determining the condition comprises determining that a duration between the first timestamp of the previously performed oxygen saturation measurement operation and a second timestamp associated with the determining of the condition to enable the oxygen saturation measurement satisfies a threshold.

The description set forth herein, in connection with the appended drawings, describes example configurations and does not represent all the examples that may be implemented or that are within the scope of the claims. The term “exemplary” used herein means “serving as an example, instance, or illustration,” and not “preferred” or “advantageous over other examples.” The detailed description includes specific details for the purpose of providing an understanding of the described techniques. These techniques, however, may be practiced without these specific details. In some instances, well-known structures and devices are shown in block diagram form in order to avoid obscuring the concepts of the described examples.

In the appended figures, similar components or features may have the same reference label. Further, various components of the same type may be distinguished by following the reference label by a dash and a second label that distinguishes among the similar components. If just the first reference label is used in the specification, the description is applicable to any one of the similar components having the same first reference label irrespective of the second reference label.

Information and signals described herein may be represented using any of a variety of different technologies and techniques. For example, data, instructions, commands, information, signals, bits, symbols, and chips that may be referenced throughout the above description may be represented by voltages, currents, electromagnetic waves, magnetic fields or particles, optical fields or particles, or any combination thereof.

The various illustrative blocks and modules described in connection with the disclosure herein may be implemented or performed with a general-purpose processor, a DSP, an ASIC, an FPGA or other programmable logic device, discrete gate or transistor logic, discrete hardware components, or any combination thereof designed to perform the functions described herein. A general-purpose processor may be a microprocessor, but in the alternative, the processor may be any conventional processor, controller, microcontroller, or state machine. A processor may also be implemented as a combination of computing devices (e.g., a combination of a DSP and a microprocessor, multiple microprocessors, one or more microprocessors in conjunction with a DSP core, or any other such configuration).

The functions described herein may be implemented in hardware, software executed by a processor, firmware, or any combination thereof. If implemented in software executed by a processor, the functions may be stored on or transmitted over as one or more instructions or code on a computer-readable medium. Other examples and implementations are within the scope of the disclosure and appended claims. For example, due to the nature of software, functions described above can be implemented using software executed by a processor, hardware, firmware, hardwiring, or combinations of any of these. Features implementing functions may also be physically located at various positions, including being distributed such that portions of functions are implemented at different physical locations. Also, as used herein, including in the claims, “or” as used in a list of items (for example, a list of items prefaced by a phrase such as “at least one of” or “one or more of”) indicates an inclusive list such that, for example, a list of at least one of A, B, or C means A or B or C or AB or AC or BC or ABC (i.e., A and B and C). Also, as used herein, the phrase “based on” shall not be construed as a reference to a closed set of conditions. For example, an exemplary step that is described as “based on condition A” may be based on both a condition A and a condition B without departing from the scope of the present disclosure. In other words, as used herein, the phrase “based on” shall be construed in the same manner as the phrase “based at least in part on.”

Computer-readable media includes both non-transitory computer storage media and communication media including any medium that facilitates transfer of a computer program from one place to another. A non-transitory storage medium may be any available medium that can be accessed by a general purpose or special purpose computer. By way of example, and not limitation, non-transitory computer-readable media can comprise RAM, ROM, electrically erasable programmable ROM (EEPROM), compact disk (CD) ROM or other optical disk storage, magnetic disk storage or other magnetic storage devices, or any other non-transitory medium that can be used to carry or store desired program code means in the form of instructions or data structures and that can be accessed by a general-purpose or special-purpose computer, or a general-purpose or special-purpose processor. Also, any connection is properly termed a computer-readable medium. For example, if the software is transmitted from a website, server, or other remote source using a coaxial cable, fiber optic cable, twisted pair, digital subscriber line (DSL), or wireless technologies such as infrared, radio, and microwave, then the coaxial cable, fiber optic cable, twisted pair, DSL, or wireless technologies such as infrared, radio, and microwave are included in the definition of medium. Disk and disc, as used herein, include CD, laser disc, optical disc, digital versatile disc (DVD), floppy disk and Blu-ray disc where disks usually reproduce data magnetically, while discs reproduce data optically with lasers. Combinations of the above are also included within the scope of computer-readable media.

The description herein is provided to enable a person skilled in the art to make or use the disclosure. Various modifications to the disclosure will be readily apparent to those skilled in the art, and the generic principles defined herein may be applied to other variations without departing from the scope of the disclosure. Thus, the disclosure is not limited to the examples and designs described herein, but is to be accorded the broadest scope consistent with the principles and novel features disclosed herein.

What is claimed is:

1. A system for detecting breathing disturbance, comprising:
a wearable ring device configured to acquire physiological data from a user, the physiological data comprising oxygen saturation data collected via photoplethysmogram (PPG) measurements from one or more light-emitting components and one or more light-receiving components of the wearable ring device, heartbeat data collected via the PPG measurements from the one or more light-emitting components and the one or more light-receiving components of the wearable ring device, heartbeat data collected via an electrocardiogram sensor of the wearable ring device, pulse wave amplitude data collected via the PPG measurements, movement data comprising an intensity of movement of the user collected via one or more accelerometers of the wearable ring device, temperature data collected via one or more temperature sensors of the wearable ring device, or some combination thereof, wherein the physiological data is collected throughout a time interval that includes one or more sleep periods of the user; and
one or more processors communicatively coupled with the wearable ring device, wherein the one or more processors are configured to:
receive the physiological data acquired via the wearable ring device via one or more electronic signals;
input the physiological data into a neural network, the neural network trained to detect breathing disturbance events based on a plurality of features within a training physiological dataset associated with a plurality of users, the plurality of features comprising a first decrease in an oxygen saturation level relative to a baseline oxygen saturation level of the plurality of users, a second decrease in a pulse wave amplitude relative to a baseline heartbeat amplitude of the plurality of users, an increase in intensity of movement relative to a baseline intensity of the plurality of users, a third decrease in temperature data relative to baseline temperature data of the plurality of users, or some combination thereof;
generate, using the neural network, a breathing disturbance metric based at least in part on the plurality of features within the physiological data during the time interval, the breathing disturbance metric associated with a probability that the user experienced a breathing disturbance event during the time interval; and
transmit an instruction to a graphical user interface (GUI) of a user device associated with the wearable ring device, the instruction configured to cause the GUI to display information associated with the breathing disturbance metric.

2. The system of claim 1, wherein the one or more processors are further configured to:
determine a condition to trigger acquisition of the physiological data from the user associated with the wearable ring device, wherein the condition corresponds to a physical state of the wearable ring device, a physiological state of the user, or both, and wherein the condition is determined based at least in part on one or more relationships between sensor data from the wearable ring device, application data, physiological data from the wearable ring device, or any combination thereof;
receive the oxygen saturation data associated with the user from the wearable ring device based at least in part on the condition; and
cause the GUI to display an indication of the oxygen saturation data for the user.

3. The system of claim 2, further comprising:
acquiring the physiological data based at least in part on a sleep state of the user, wherein determining the condition comprises:
detecting the sleep state of the user.

4. The system of claim 1, wherein the breathing disturbance metric comprises a probability of an apnea event, a probability of a hypopnea event, a probability of an oxygen desaturation event, a probability of a respiratory effort-related arousal event, or some combination thereof.

5. The system of claim 1, wherein the information associated with the breathing disturbance metric comprises a breathing disturbance index, an apnea-hypopnea index, an oxygen desaturation index, or some combination thereof.

6. The system of claim 1, wherein the one or more processors are further configured to:
transmit an instruction to the GUI to cause the GUI to display a timeline associated with the one or more sleep periods of the user, wherein the timeline comprises one or more breathing disturbance events and a timestamp associated with the one or more breathing disturbance events.

7. The system of claim 1, wherein the one or more processors are further configured to:
select the neural network from a set of neural networks, wherein each neural network of the set of neural networks is associated with one or more combinations of physiological data.

8. The system of claim 1, wherein the one or more processors are further configured to:
perform a preprocessing of the training physiological dataset and of the physiological data, wherein the preprocessing comprises filtering the training physiological dataset and the physiological data, normalizing the training physiological dataset and the physiological data, resampling the training physiological dataset and the physiological data, padding the training physiological dataset and the physiological data, or some combination thereof; and
train the neural network based at least in part on the preprocessed training physiological dataset.

9. The system of claim 8, wherein training the neural network comprises:
determining one or more parameter values associated with the neural network based at least in part on an error between one or more ground-truth labels associated with the preprocessed training physiological dataset and one or more predicted labels output from the neural network.

10. The system of claim 1, wherein, to generate the breathing disturbance metric, the one or more processors are further configured to:
generate the breathing disturbance metric periodically during the time interval, wherein a periodicity of the breathing disturbance metric is uniform or irregular.

11. The system of claim 1, wherein, to input the physiological data into the neural network, the one or more processors are further configured to:
input an auditory measurement into the neural network, the auditory measurement comprising a measurement of an ambient sound level during the time interval, wherein the plurality of features within the training physiological dataset further comprise an increase in ambient sound level relative to a baseline ambient sound level.

12. The system of claim 11, wherein the one or more processors are further configured to:
perform the auditory measurement during the time interval via a microphone of the wearable ring device, via a charger of the wearable ring device, or via the user device.
